(19) United States
(12) Patent Application Publication
(10) Pub. No.: US 2022/0105124 A1
(43) Pub. Date: Apr. 7, 2022

(54) METHODS FOR TARGETED TREATMENT AND PREDICTION OF PATIENT SURVIVAL IN CANCER

(71) Applicant: University of Florida Research Foundation, Incorporated, Gainesville, FL (US)

(72) Inventors: David Tran, Gainesville, FL (US); Son Bang Le, Gainesville, FL (US)

(21) Appl. No.: 17/428,749

(22) PCT Filed: Feb. 6, 2020
(86) PCT No.: PCT/US2020/017073

§ 371 Date: Aug. 5, 2021

Related U.S. Application Data
(60) Provisional application No. 62/802,653, filed on Feb. 7, 2019.

(51) Int. Cl. A61K 31/7105; A61K 35/15; A61K 31/593; A61K 31/167; A61K 38/15; A61K 31/18; A61K 31/4045; A61K 31/4406; A61K 31/19; A61K 31/519; A61K 31/506; A61K 31/155; A61K 31/4709; A61K 31/409; A61K 31/4545; A61K 38/17; A61P 35/00; C12Q 1/6886; G01N 33/574

(52) U.S. Cl. CPC A61K 31/7105; C12Q 2600/158; A61K 31/593; A61K 31/167; A61K 38/15; A61K 31/18; A61K 31/4045; A61K 31/4406; A61K 31/19; A61K 31/519; A61K 31/506; A61K 31/155; A61K 31/4709; A61K 31/409; A61K 31/4545; A61K 38/179; A61P 35/00; C12Q 1/6886; G01N 33/574; A61K 35/15

(57) ABSTRACT

Provided herein are methods of assessing, detecting, monitoring the presence, or monitoring progression of cancer in a subject, or assessing or predicting prognosis or survival of a subject having cancer. Also provided are methods of treating cancer based on an increase in the expression of one or more top master regulators of a cancer.

### FEDERALLY SPONSORED RESEARCH OR DEVELOPMENT

This invention was made with government support under K08 CA160824 awarded by NIH/NCI. The government has certain rights in the invention.

### BACKGROUND

In 2018, the American Cancer Society estimated that there were 856,370 new cases of cancer in men and 878,980 new cases of cancer in women. Additionally, there were an estimated 323,630 cancer deaths in men and 286,010 cancer deaths in women. The leading sites of new cancer in men were prostate (19%), lung and bronchus (14%), and colon and rectum (9%). The leading sites of new cancer in women were breast (30%), lung and bronchus (13%), and colon and rectum (7%).

There remains a need to understand and treat cancer and to identify new targets for cancer therapeutics.

### SUMMARY

Provided are methods of assessing, detecting, monitoring the presence, or monitoring progression of cancer in a subject, or assessing or predicting prognosis or survival of a subject having cancer. The methods comprise measuring the expression level of at least one master regulator in a sample from the subject and comparing the expression level with the expression level of a corresponding master regulator gene in a healthy reference sample. An increase in the expression level of the at least one master regulator in the subject relative to the expression level of the corresponding master regulator in a healthy reference sample indicates the possible presence of cancer in the subject, an increase or risk of increase in cancer progression in the subject, a possible increased risk of developing cancer in the subject, a poor prognosis, or reduced predicted survival time for the subject.

Described herein are methods of treating cancer based on an increase in the expression of one or more top master regulators (also termed “master of death” or “master regulator or poor prognosis”). In some embodiments, an increase in the expression level of a top master regulator is a statistically significant increase in expression. In some embodiments, an increase in the expression level of a top master regulator is an increase of at least 10%, at least 20%, at least 25%, at least 30%, at least 40%, or at least 50%. In some embodiments, an increase in the expression level of a top master regulator is an increase of at least 1.5×, at least 2×, at least 2.5×, at least 3×, at least 4× or at least 5×.

An increase in the expression of one or more top master regulators of a cancer in a subject is indicative of poor prognosis for the subject. The described methods can be used to diagnosis cancer in a subject. The described methods can be used diagnose poor prognosis in a subject having cancer. The described methods can be used to guide or suggest treatments or changes in treatment of cancer. The described methods can be used to diagnose or provide guidance for treatment or changes in treatment of an individual subject. The described methods can be used to diagnose or provide guidance for treatment or changes in treatment of an individual subject based on the expression profile of one or more top master regulators. In some embodiments, the subject in a human patient.

The master regulators can be grouped according to cancer type or according to certain cellular processes. In some embodiments, elevated expression of one or more of the top regulators of death associated with any of the cancers of Tables 2 and 3 is indicative of poor prognosis for the that cancer. In some embodiments, elevated expression of one or more of the top regulators of death associated with the cellular processes of as in FIG. 17 is indicative of poor prognosis. In some embodiments, an indication of poor prognosis indicates the subject having the cancer should be treated more aggressively. In some embodiments, an indication of poor prognosis indicates the subject should be treated with one or more therapeutics known to have effectiveness in treating cancers having with a similar master regulator expression profile, i.e., having increase expression of one or more of the same top master regulators.

We have observed expression of the top master regulators is several different cancers is predictive of poor prognosis. Classes or types of cancer that effect similar cells or tissues or appear similar morphologically or histologically may be different with respect to gene expression. Similarly, cancers that have certain detectable genomic mutations may not express the mutant gene(s). For this reason, treatment cancer based expression of master regulators can better predict treatment effectiveness and prognosis. Further, such analyses can be performed on samples from individual subjects, allowing for improved diagnosis and treatment base on the expression of master regulators in the individual subject. The described methods also provide for correlation of treatment of the cancer with the master regulator expression profile of the cancer in the individual.

In some embodiments, the methods are used to assess whether a subject has a poor survival prognosis for cancer comprising: analyzing the expression level of at least one master regulator in a sample from the subject, wherein an increase in the expression level of the at least one master regulator relative to the expression level of the corresponding master regulator in a healthy reference sample, is indicative that the subject has a poor survival prognosis for the cancer. In some embodiments, the master regulator is a gene as in Tables 2 and 3.

In some embodiments, one or more cancer therapies is administered to a subject identified as having a poor prognosis or reduced predicted survival time.

### BRIEF DESCRIPTION OF THE DRAWINGS

Having thus described the invention in general terms, reference will now be made to the accompanying drawings, which are not necessarily drawn to scale.

FIG. 1 illustrates leading sites of new cancer cases and deaths—2018 estimates.

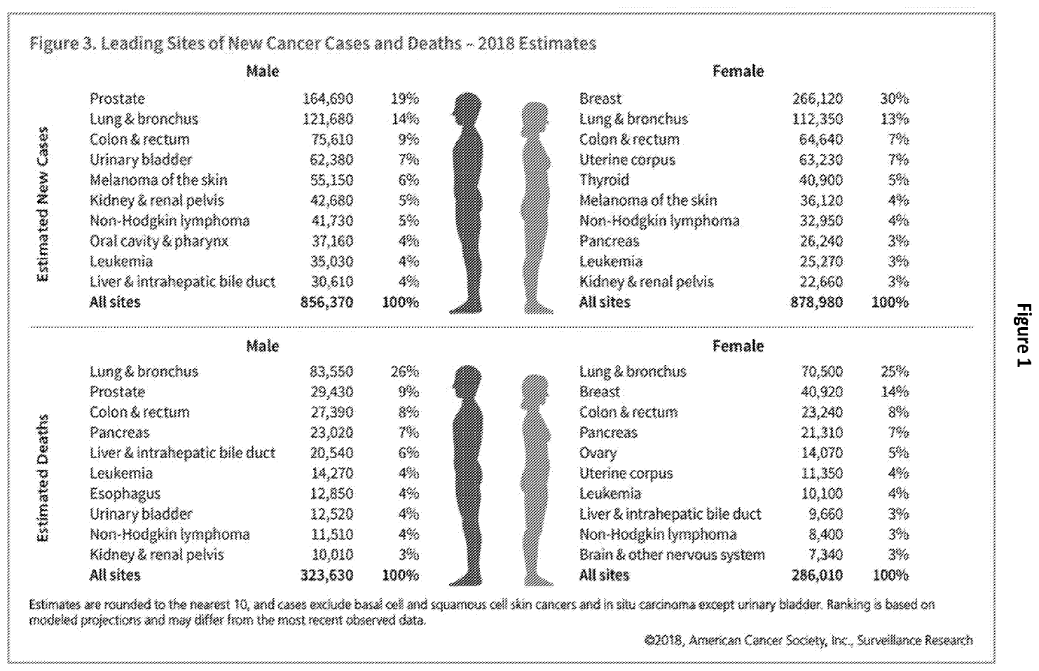

FIG. 2 illustrates the top master regulators of poor prognosis in lung adenocarcinoma.

FIG. 3 illustrates the top master regulators of poor prognosis in lung squamous cell carcinoma.

FIG. 4 illustrates the top master regulators of poor prognosis in breast invasive carcinoma.

FIG. 5 illustrates the top master regulators of poor prognosis in prostate adenocarcinoma.

FIG. 6 illustrates the top master regulators of poor prognosis in colon and rectum adenocarcinoma.

FIG. 7 illustrates the top master regulators of poor prognosis in pancreatic adenocarcinoma.

FIG. 8 illustrates the top master regulators of poor prognosis in liver hepatocellular carcinoma.

FIG. 9 illustrates the top master regulators of poor prognosis in acute myeloid leukemia.

FIG. 10 illustrates the top master regulators of poor prognosis in ovarian serous cystadenocarcinoma.

FIG. 11 illustrates the top master regulators of poor prognosis in glioblastoma multiforme.

FIG. 12 illustrates the top master regulator of poor prognosis for each cancer subtype.

FIG. 13A illustrates the frequency of master regulators of poor prognosis.

FIG. 13B illustrates the most frequent master regulators of poor prognosis.

FIG. 14 illustrates 4 cancer groups by master regulators of poor prognosis.

FIG. 15 illustrates heatmap of common master regulators of poor prognosis. nScore values of Masters of death that present in at least two cancer subtypes.

FIG. 16 illustrates frequency of pathways of poor prognosis in different cancer subtypes.

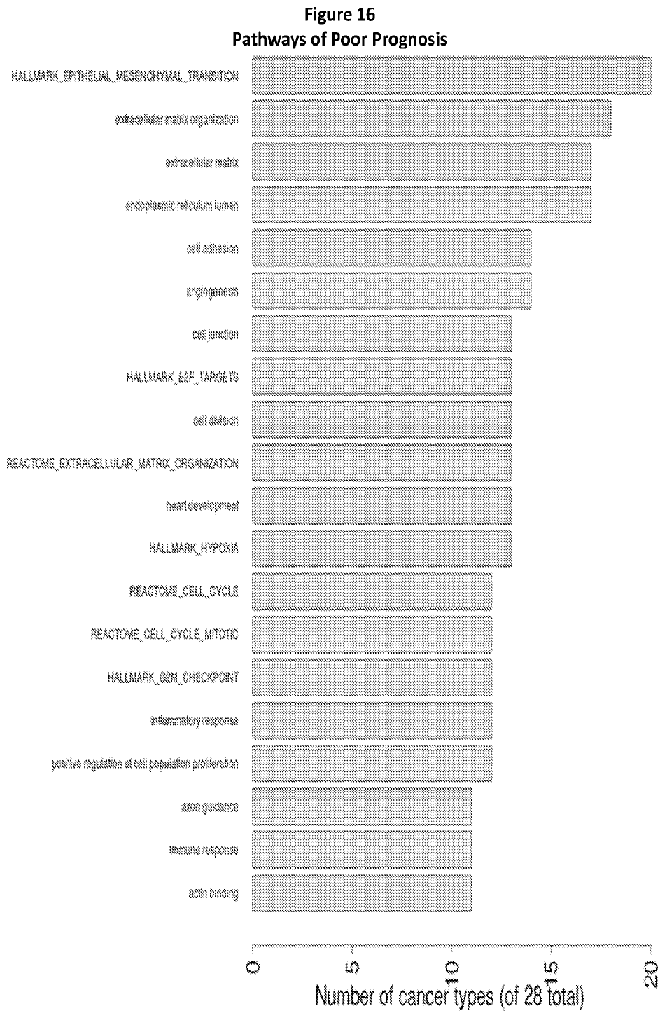

FIG. 17 illustrates the master regulators in selected pathways of poor prognosis.

FIG. 18A-B illustrate the effect of knockdown of VDR expression on MYD88, CLCF1, LIF, and OSMR in GBM cell lines. The indicated GBM cells were transfected with empty vector (EV; left bar in each gene) or short hairpin VDR (shVDR; right bar for each gene). shVDR inhibits expression of VDR

FIG. 18C illustrates the effect of knockdown of VDR expression on MYD88, CLCF1, LIF, and OSMR in a GBM cell line. The indicated GBM cells were transfected with empty vector (EV; left bar in each gene) or short hairpin VDR (shVDR; right bar for each gene). shVDR inhibits expression of VDR.

FIG. 19A-B illustrate the effect of knockdown of VDR expression on various genes in GBM cell lines. The indicated GBM cells were transfected with empty vector (EV; left bar in each gene) or short hairpin VDR (shVDR; right bar in each gene). shVDR inhibits expression of VDR.

FIG. 19C-D illustrate the effect of knockdown of VDR expression on various genes in GBM cell lines. The indicated GBM cells were transfected with empty vector (EV; left bar in each gene) or short hairpin VDR (shVDR; right bar in each gene). shVDR inhibits expression of VDR.

FIG. 20 illustrates genes in the VDR network.

FIG. 21A illustrates correlation of MYBL2 expression with survival in renal and liver cancer patients.

FIG. 21B illustrates correlation of FOXM1 expression with survival in renal and pancreatic cancer patients.

FIG. 21C illustrates correlation of PTTG1 expression with survival in renal and liver cancer patients.

### DEFINITIONS

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Expression of master regulator genes in cancer drive bad cancer behavior or poor prognosis of the cancer. Poor prognosis can include, but is not limited to, poor response to typical cancer treatment, aggressive cancer growth, increased metastasis, and/or reduced survival time. Identification of poor prognosis in a subject can be used to diagnose and/or prescribe treatment. Such treatment can include, but is not limited to, master regulator-specific treatment and/or more aggressive treatment. Master regulator-specific treatment includes treatments, including adjuvants, known to be effective in treating similar cancers in other subjects expressing the same master regulator gene(s). As an example, subjects having increased expression of VDR or VDR-related genes may be given vitamin D.

A “sample” comprises any tissue or material isolated from a subject, such as a patient. The sample may contain cellular and/or non-cellular material from the subject, and may contain any biological material suitable for detecting a desired biomarker, such a DNA or RNA. The sample can be isolated from any suitable biological tissue or fluid such as, but not limited to, a tissue or blood. A sample may be treated physically, chemically, and/or mechanically to disrupt tissue or cell structure, thus releasing intracellular components into a solution which may further contain enzymes, buffers, salts, detergents and the like, which are used to prepare the sample for analysis.

The “epithelial-mesenchymal transition” (EMT) is a process by which epithelial cells lose gene expression patterns and behaviors characteristic of epithelial cells, including adhesion and apical-basal polarity, and begin to look and behave like, and express genes typical of, mesenchymal cells, gaining migratory and invasive properties. EMT has also been shown to occur in the initiation of metastasis in cancer progression.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### DETAILED DESCRIPTION

Various embodiments of the inventions now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

Described are detailed reference gene networks for major types of cancers based on genome-wide expression profiles in the Cancer Genome Atlas, using the GeneRep algorithm. Reference gene networks provide a foundational framework on which to understand the mechanism of cancer development on a global scale and to identify master regulators and therapeutic development. Master regulators are genes at the top of a gene network that can alter the expression of downstream genes in a network. The described networks contain the largest number of connections with the highest statistical confidence.

Using nScore algorithms applied to survival time, we have identified master regulators of poor prognosis in a number of different cancers. Poor prognosis refers to reduced predicted survival time. These will be critical in understanding the global properties of cancer cells across multiple major cancers in humans and serve as a foundation for diagnostic and therapeutic development.

Described are methods assessing, detecting, monitoring the presence, or monitoring progression of cancer in a subject, or assessing or predicting prognosis or survival of a subject having cancer. The methods comprise

a) obtaining or having obtained a sample from a subject

b) measuring or having measured the expression level of at least one master regulator in the sample; and

c) comparing the expression level with the expression level of a corresponding master regulator gene in a healthy reference sample;

wherein an increase in the expression level of the at least one master regulator in the subject relative to the expression level of the corresponding master regulator in the healthy reference sample indicates the possible presence of cancer in the subject, an increase or risk of increase in cancer progression in the subject, an increased risk of developing cancer in the subject, a poor prognosis, or decreased predicted survival time for the subject. The methods can be used to suggest treatments or changes in treatment of the subject.

In some embodiments, the methods are used to assess whether a subject has a decreased predicted survival time for cancer comprising: measuring the expression level of at least one master regulator in a sample from the subject, wherein an increase in the expression level of the at least one master regulator relative to the expression level of the corresponding master regulator in a healthy reference sample, is indicative that the subject has a poor survival prognosis for the cancer. In some embodiments, the master regulator is a gene as in Tables 2 and 3.

In some embodiments, measuring expression levels of one or more of the master regulators of Tables 2 and 3 can be used to monitor cancer growth in a subject.

In some embodiments, expression levels of 2, 3, 4, 5, 6, 7, 8, 9, 10, 11, 12, 13, 14, 15, 16, 17, 18, 19, or 20 master regulators in a subject sample are measured and compared with the expression level of the corresponding master regulators in a healthy reference (control) sample.

In some embodiments, master regulators are selected based on the cancer type. The top 20 master regulators for several cancer types are shown in Tables 2 and 3.

In some embodiments, master regulators are selected based of the occurrence of the master regulator in several cancer types. Master regulators that have increase expression in several cancer types may be selected from the group consisting of: MYBL2, MYBL2, PTTG1, FOXM1, E2F7, CDK1, UHRF1, TRIP13, TRIM29, HDAC7, ARNTL2, AEBP1, or ACTL6A.

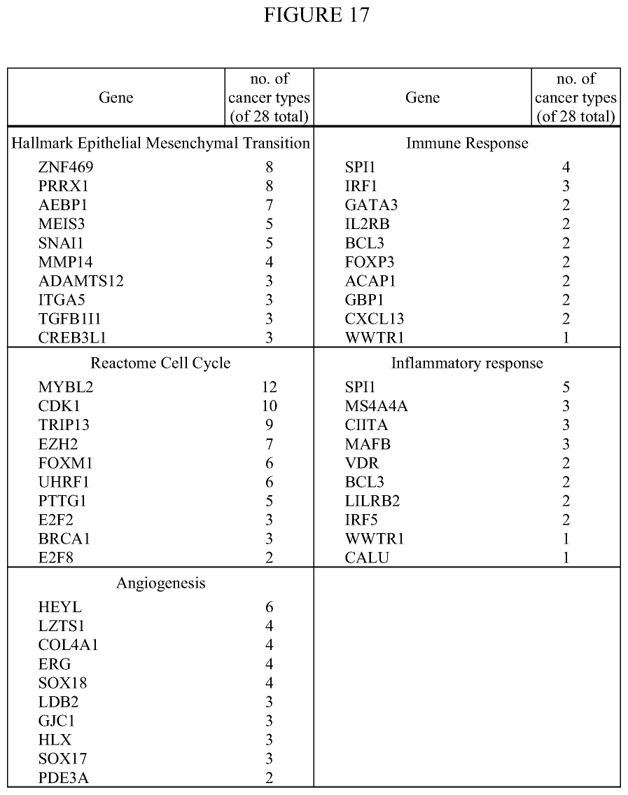

In some embodiments, master regulators can be grouped based on their association with certain cellular processes, such as, but not limited to, cell cycle, epigenetic/chromosome remodeling, Epithelial Mesenchymal Transitions (EMT), immune/development, angiogenesis, immune response, and inflammatory response. Detection of increased expression of one or more of the master regulators in any of the groups of FIG. 17 is indicative of poor prognosis. Detection of increased expression of one or more of the master regulators in any of the groups of FIG. 17 indicates treatment with one or more therapeutics known to have effectiveness in treating cancers having with a similar master regulator expression profile is recommended.

Methods of determining gene expression in a sample can be performed using methods know in the art. Such methods included, but are not limited to, nucleotide amplification assays (including but not limited to PCR, RT-PCR, serial analysis of gene expression, and differential display), RNA sequencing, microarray technologies, proteomics, HPLC, Western electrophoresis.

Monitoring cancer growth can be used to direct treatment of the cancer, wherein an increase in expression of one or more master regulators indicates poor prognosis or decreased predicted survival time. In some embodiments, one or more cancer therapies is administered to a subject identified as having a poor prognosis or decreased predicted survival time.

### Treatment

In some embodiments, we describe methods of treating cancer comprising inhibiting one or more master regulators. Inhibiting one or more master regulators can comprise using or administering one or more master regulator antagonists or inhibitors. A master regulator can be inhibited at the gene level, such as by using or administering RNA interference agents or antisense oligonucleotides to inhibit expression of the gene. The master regulators can be inhibited at the protein level, such as by using or administering an immunotherapy composition that binds to the master regulator protein and inhibits activity of the protein or by using or administering a small molecule drug known to inhibit activity of the master regulator protein. In some embodiments, we described methods of treating cancer comprising using or administering an immunotherapy composition against a master regulator protein or a combination of master regulator proteins. An immunotherapy composition can comprise one or more antibodies having affinity for one or more master regulators. An antibody can be, but is not limited to, an immunoglobulin, an immunoglobulin fragment having affinity for the master regulator, a chimeric antibody, a bispecific antibody, an antibody conjugate, or the like.

In some embodiments, an immunotherapy composition comprises a peptide formulation derived from a master regulator of poor prognosis. The peptide can be an immunogenic fragment of a master regulator protein. The peptide can be combined with an immune stimulating adjuvant. The immunotherapy composition can be administered locally (e. g., subcutaneously) or systemically (e. g., intravenously) with or without the presence of adjuvant. The immunotherapy composition can be used to stimulate the immune system to develop an immune reaction specifically against the master regulator of poor prognosis. Development of an immune reaction can eliminate or aid in eliminating cancer cells expressing the master regulator of poor prognosis.

In some embodiments, we describe methods of treating cancer comprising using or administering one or more small molecule drugs to inhibit activity of a master regulator protein or a combination of master regulator proteins. Small molecule drugs include, but are not limited to those in Table A.

TABLE A
Master regulators and known therapies
directed at the master regulator.

| | Inhibitors, drugs, or hormones that |
|---|---|
| Master regulators | can block or mitigate abnormal signals |
| (Cancers) | emanating from these master regulators |
| VDR (GBM, glioma, | Vitamin D: In GBM cells with high VDR |
| AML) | expression, which has abnormal signaling |
| | leading to higher Sox2 expression and |
| | driving cancer stem cell growth. Treating |
| | cells with vitamin D reduces this abnormal |
| | signal from VDR, leading to lower Sox2, |
| | while VDR expression is relatively |
| | unaffected. |
| CDK1 (lung adenocarcinoma) | CDK1/2 inhibitors, e.g. Flavopiridol |
| HDAC7 (Lung squamous cell | HDAC inhibitors, e.g. vorinostat, |
| carcinoma, colon & rectal | romidepsin, belinostat, panobinostat, |
| adenocarcinoma, GBM, | entinostat, valproic acid |
| AML) | |
| YAP1 (Pancreatic adeno- | Yap1 inhibitors: Vereporfin, CA3, or |
| carcinoma) | drugs that targeting downstream or |
| | in the pathways of YAP1, e.g., |
| | Trametinib, dasatinib, metformin |
| HDAC2 (hepatocellular | HDAC inhibitors, e.g. vorinostat, |
| carcinoma) | romidepsin, belinostat, panobinostat, |
| | entinostat, valproic acid |
| SMAD7 (Lung squamous | Mongersen or the TGFbeta pathway |
| cell carcinoma) | inhibitors, e.g. galunisertib, AVID200 |

In some embodiments, we describe methods of treating cancer comprising using or administering one or more antisense oligonucleotides or RNA interference agents to knock down expression of a master regulator gene or a combination of master regulator genes. An antisense oligonucleotide is a single-stranded oligonucleotide having a nucleobase sequence that permits hybridization to a corresponding region or segment of a target nucleic acid. An RNA interference agent is an oligonucleotide that mediates the targeted cleavage of an RNA transcript in a sequence specific manner via an RNA-induced silencing complex (RISC) pathway.

In some embodiments, we describe methods of treating cancer comprising using or administering a combination of one or more master regulator antagonists or inhibitors.

In some embodiments, treating a cancer of any of the cancer types in Tables 2 and 3 comprises administering one or more inhibitors of at least one master regulator identified as a top 20 master regulator for the cancer type as indicated in Tables 2 and 3.

### EXAMPLES

### Example 1: Identifying Masters Regulators of Poor Prognosis Using GeneRep Algorithm

We analyzed the cancers in Table 1 based on genome-wide expression profiles in the Cancer Genome Atlas, using the GeneRep algorithm. A method of determining the master regulators of a particular cancer is GeneRep/nSCORE described in WO-2018/069891 which is incorporated by reference in its entirety.

TABLE 1

| Study Abbreviation | Cancer Type |
|---|---|
| LAML | Acute Myeloid Leukemia |
| ACC | Adrenocortical carcinoma |
| BLCA | Bladder Urothelial Carcinoma |
| LGG | Brain Lower Grade Glioma |
| BRCA | Breast invasive carcinoma |
| CESC | Cervical squamous cell carcinoma and |
| | endocervical adenocarcinoma |
| CHOL | Cholangiocarcinoma |
| LCML | Chronic Myelogenous Leukemia |
| COAD | Colon adenocarcinoma |
| CNTL | Controls |
| ESCA | Esophageal carcinoma |
| FPPP | FFPE Pilot Phase II |
| GBM | Glioblastoma multiforme |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LIHC | Liver hepatocellular carcinoma |
| LUAD | Lung adenocarcinoma |
| LUSC | Lung squamous cell carcinoma |
| DLBC | Lymphoid Neoplasm Diffuse Large B-cell |
| | Lymphoma |
| MESO | Mesothelioma |
| MISC | Miscellaneous |
| OV | Ovarian serous cystadenocarcinoma |
| PAAD | Pancreatic adenocarcinoma |
| PCPG | Pheochromocytoma and Paraganglioma |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THYM | Thymoma |
| THCA | Thyroid carcinoma |
| UCS | Uterine Carcinosarcoma |
| UCEC | Uterine Corpus Endometrial Carcinoma |
| UVM | Uveal Melanoma |

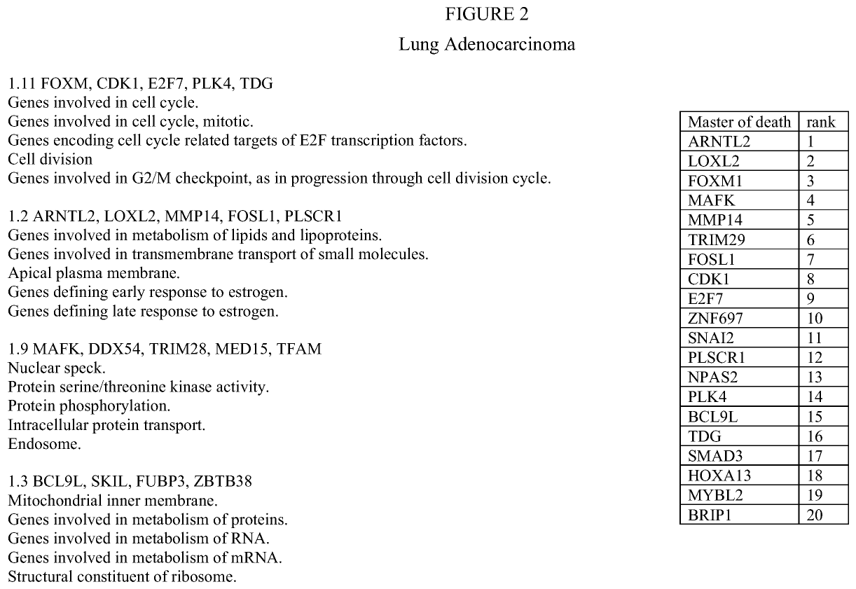

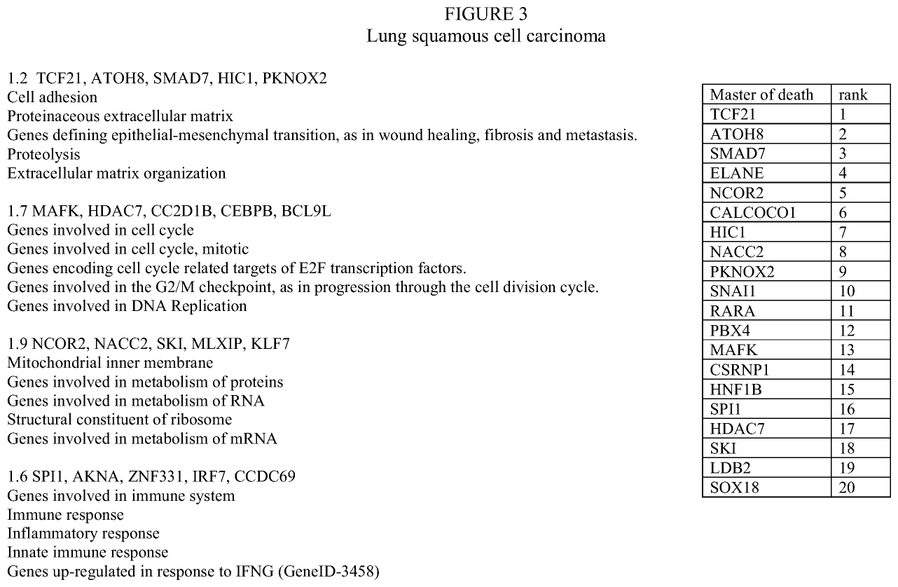

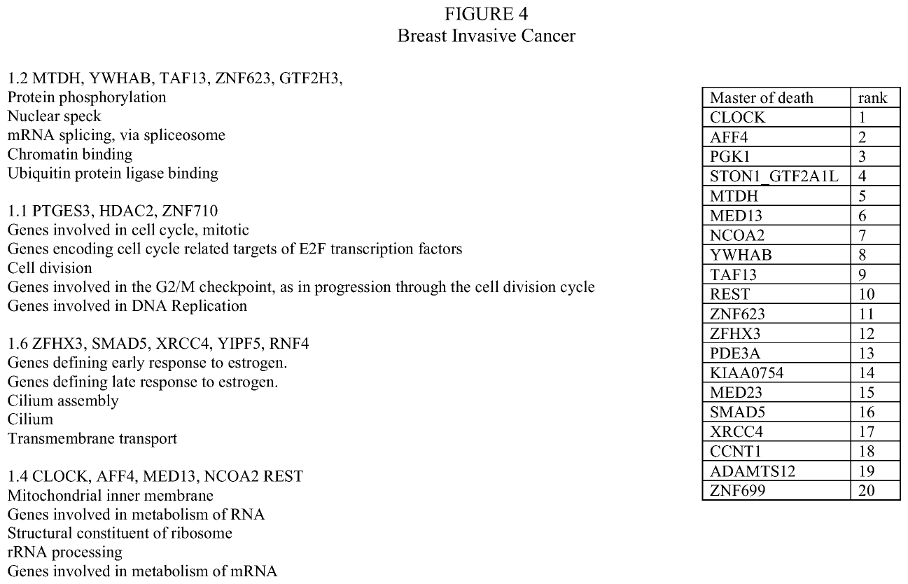

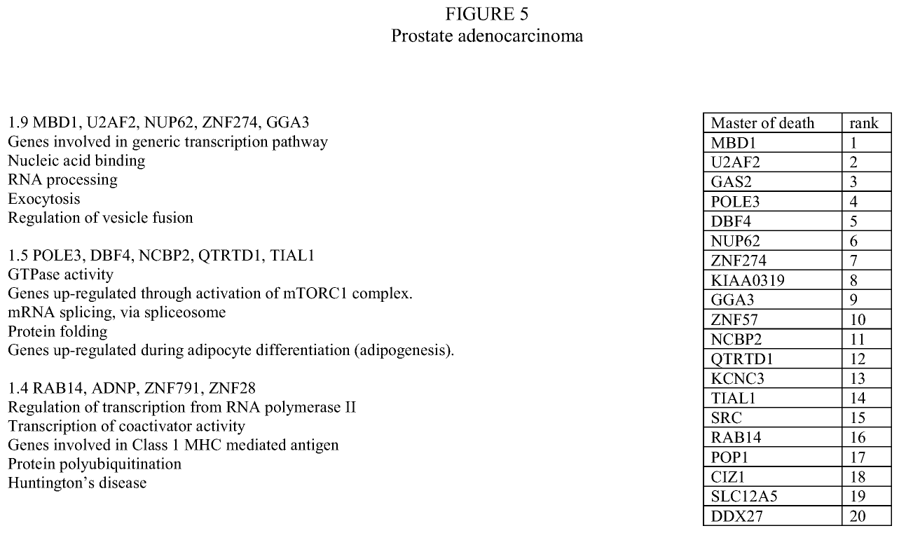

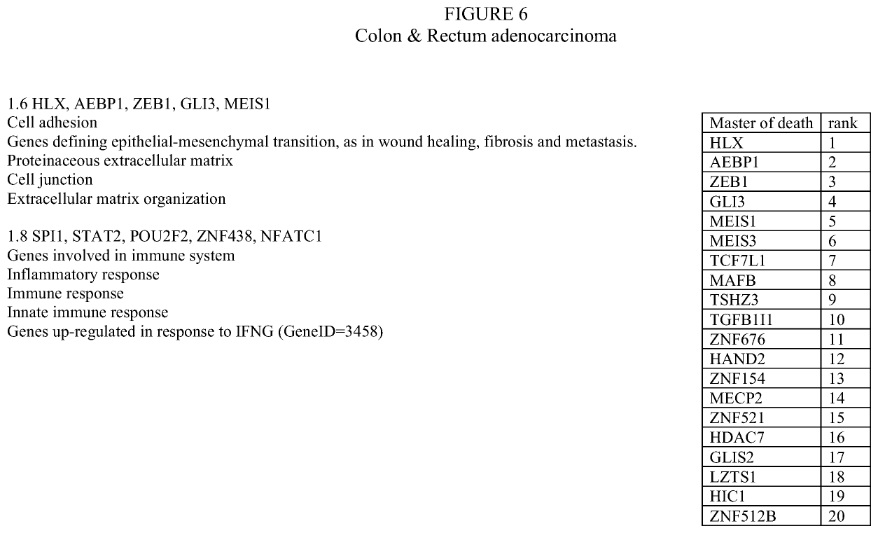

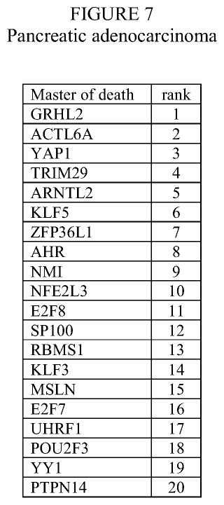

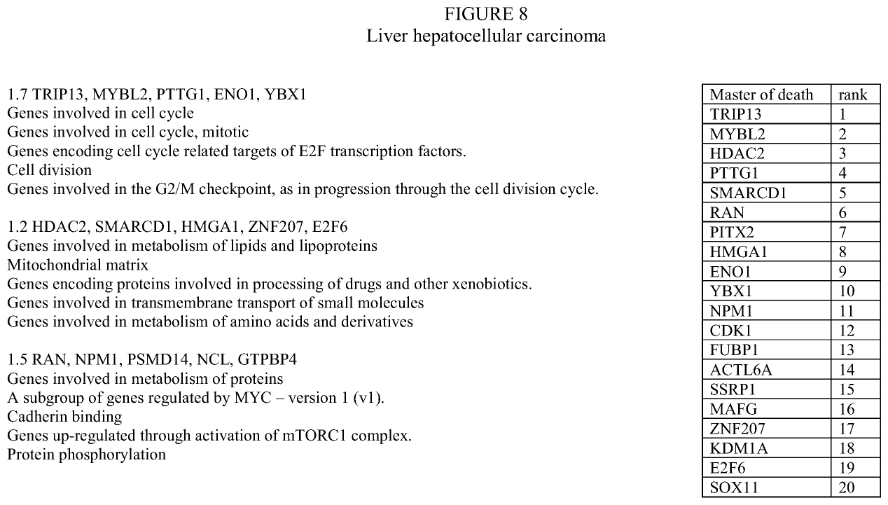

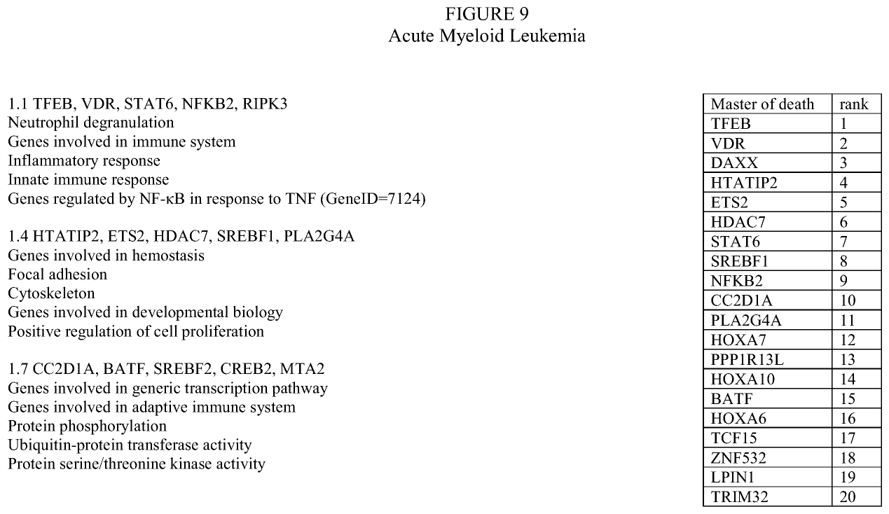

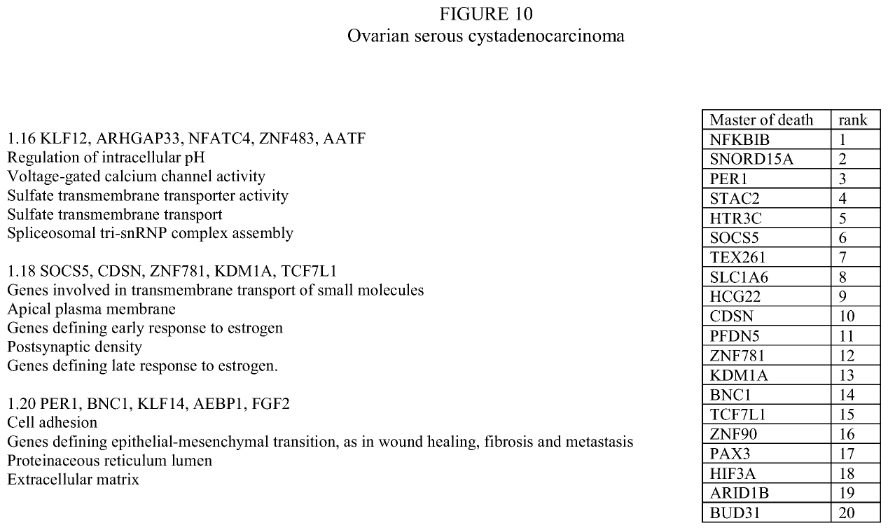

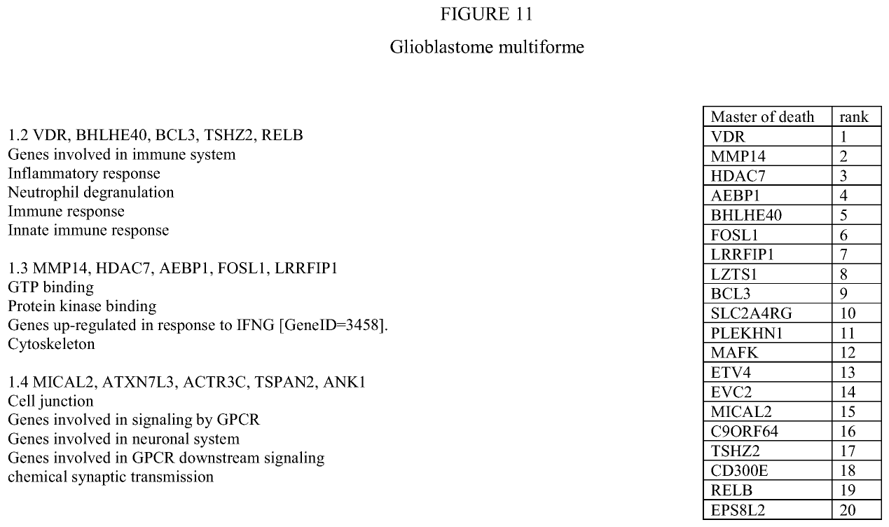

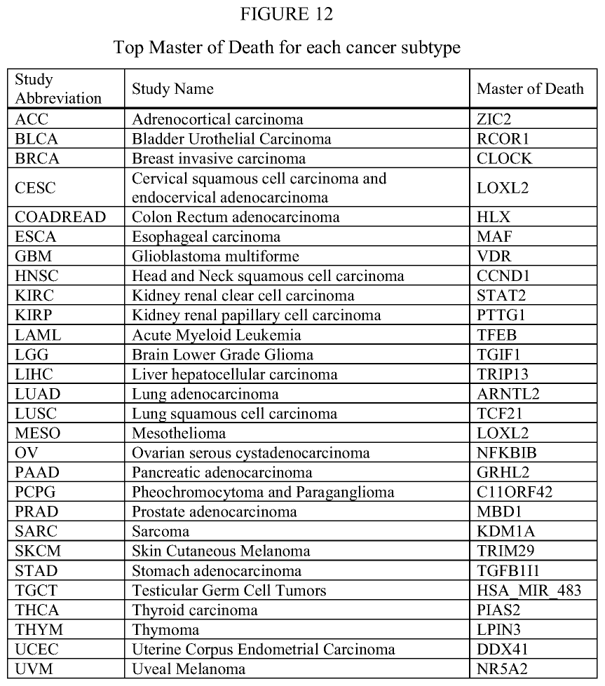

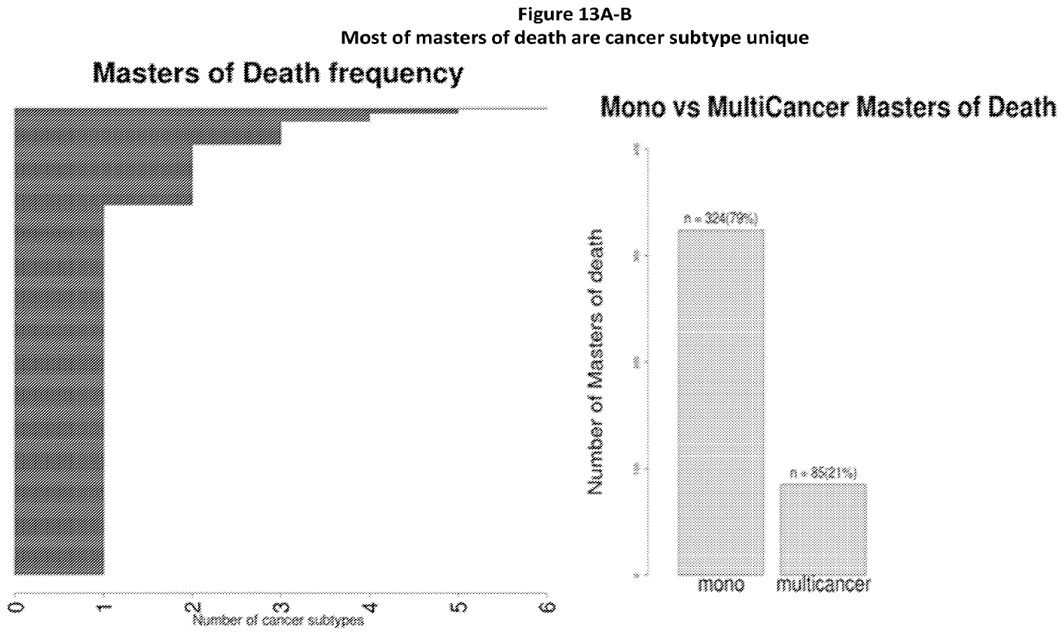

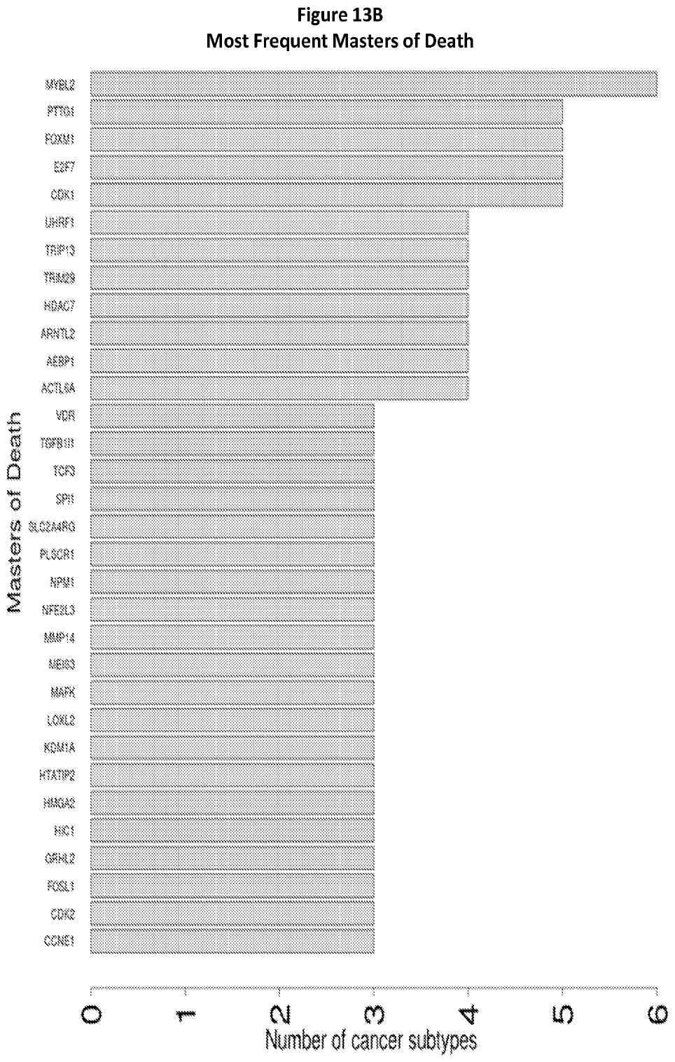

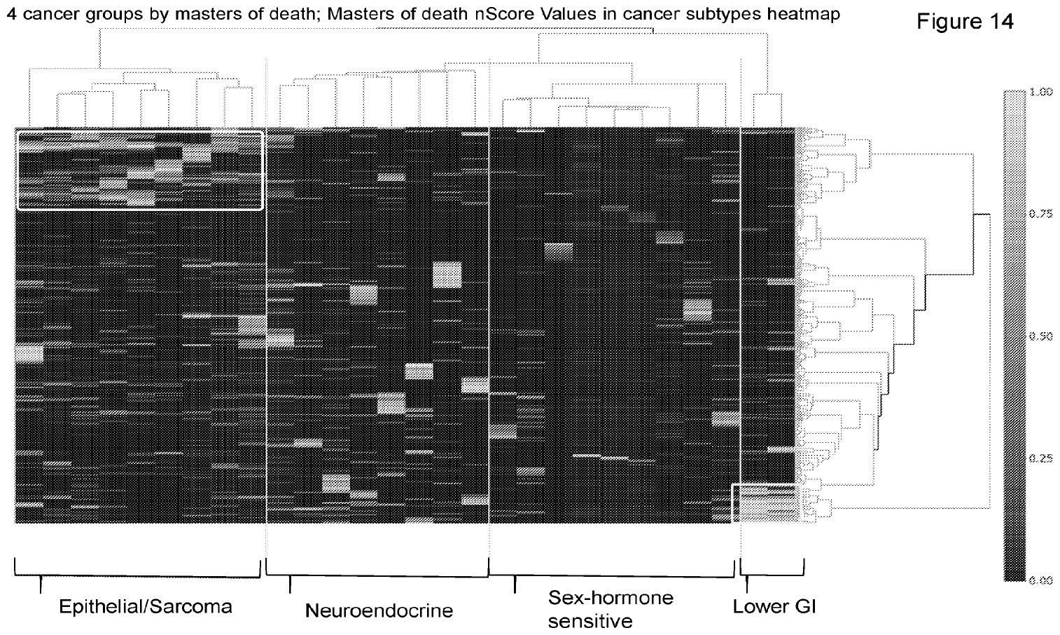

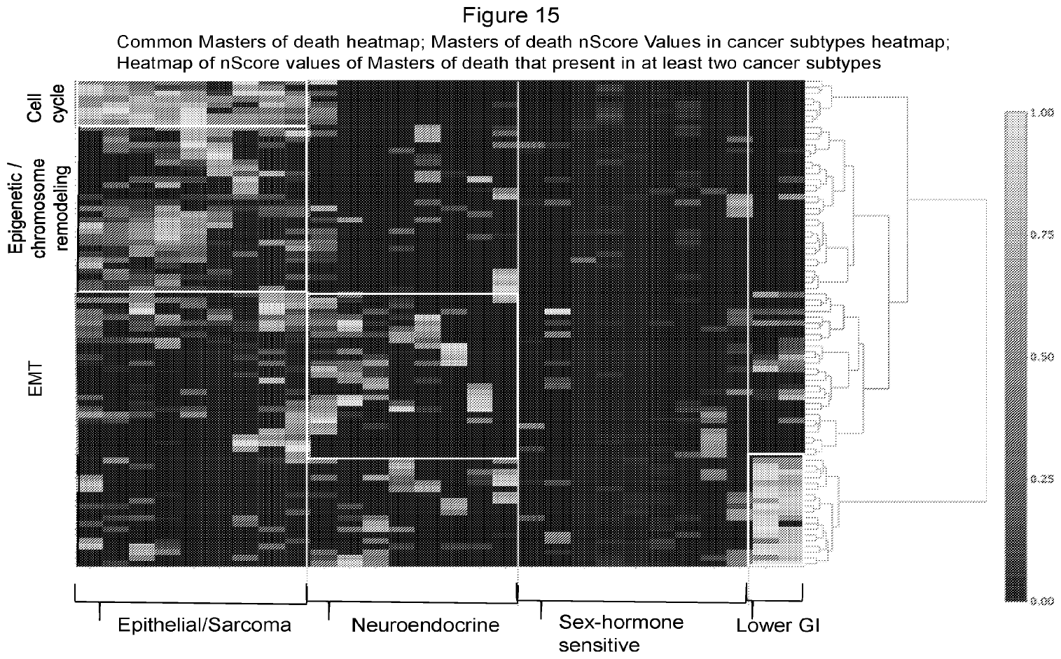

Results from our study are described in Tables 2 and 3 below and FIGS. 2-15.

TABLE 2
Top 20 master regulator genes (masters of death), labeled 1-20, for various cancer types.
Cancer types corresponding to the indicated abbreviations are listed in Table 1.

| | Cancer type | | | | | | | | | | | | | | |
|---|---|---|---|---|---|---|---|---|---|---|---|---|---|---|---|
| gene | acc | blca | brca | cesc | coadread | esca | gbm | hnsc | kirc | kirp | laml | lgg | lihc | luad | lusc |
| MYBL2 | 15 | | | | | | | | 7 | 3 | | | 2 | | |
| FOXM1 | 2 | | | | | | | | 16 | 4 | | | | 3 | |
| CDK1 | 10 | | | | | | | | | 2 | | | 12 | 10 | |
| PTTG1 | | | | | | | | | 8 | 1 | | | 4 | | |
| E2F7 | | | | | | | | | | 9 | | 9 | | 11 | |
| UHRF1 | 17 | | | | | | | | | 17 | | | | | |
| TRIP13 | 9 | | | | | | | | | 16 | | | 1 | | |
| AEBP1 | | | | | 2 | | 4 | | | | | | | | |
| HDAC7 | | | | | 16 | | 3 | | | | 6 | | | | 17 |
| ACTL6A | | | | | | | | 19 | | | | | 14 | | |
| ARNTL2 | | | | | | | | | | | | 16 | | 1 | |
| TRIM29 | | | | | | | | | | | | | | 8 | |
| HMGA2 | 11 | | | | | | | 3 | | | | | | | |
| TCF3 | 7 | | | | | | | | 15 | | | | | | |
| LOXL2 | | | | 1 | | | | | | | | | | 2 | |
| MEIS3 | | | | | 6 | | | | | | | | | | |
| TGFB1I1 | | | | | 10 | | | | | | | | | | |
| HIC1 | | | | | 19 | | | | | | | | | | 7 |
| SPI1 | | | | | | 16 | | | | | | | | | 16 |
| FOSL1 | | | | | | | 6 | 9 | | | | | | 9 | |
| MMP14 | | | | | | | 2 | | | | | | | 5 | |
| VDR | | | | | | | 1 | | | | 2 | 13 | | | |
| MAFK | | | | | | | 12 | | | | | | | 4 | 13 |
| SLC2A4RG | | | | | | | 10 | | | | | 17 | | | |
| NPM1 | | | | | | | | 18 | | 18 | | | 11 | | |
| CCNE1 | | | | | | | | | 4 | 5 | | | | | |
| CDK2 | | | | | | | | | | 19 | | 4 | | | |
| HTATIP2 | | | | | | | | | | | 4 | 19 | | | |
| NFE2L3 | | | | | | | | | | | | 10 | | | |
| PLSCR1 | | | | | | | | | | | | 15 | | 14 | |
| KDM1A | | | | | | | | | | | | | 18 | | |
| FOXD1 | 12 | | | | | | | | | | | | | | |
| EZH2 | 5 | | | | | | | | | | | | | | |
| PLK4 | 16 | | | | | | | | | | | | | 16 | |
| DNMT1 | 3 | | | | | | | | | | | | | | |
| ETV4 | 4 | | | | | | 13 | | | | | | | | |
| PCGF6 | 20 | | | | | 12 | | | | | | | | | |
| PPRC1 | 14 | | | | | | | | | | | | | | |
| ATF6 | | 7 | | | | | | | | | | | | | |
| HEYL | | 14 | | | | | | | | | | | | | |
| OTX1 | | 18 | | | | | | | | | | 14 | | | |
| SSRP1 | | 11 | | | | | | | | | | | 15 | | |
| BNC1 | | 20 | | | | | | | | | | | | | |
| ZNF521 | | 10 | | | 15 | | | | | | | | | | |
| ZNF532 | | 19 | | | | | | | | | 18 | | | | |
| REST | | | 12 | | | | | | | | | 20 | | | |
| KLF17 | | | | 9 | | | | | | | | | | | |
| LIF | | | | 4 | | | | | | | | | | | |
| NCOR2 | | | | 17 | | | | | | | | | | | 5 |
| SALL2 | | | | 8 | | | | | | | | | | | |
| HAND2 | | | | | 12 | | | | | | | | | | |
| LZTS1 | | | | | 18 | | 8 | | | | | | | | |
| TCF7L1 | | | | | 7 | | | | | | | | | | |
| TSHZ3 | | | | | 9 | | | | | | | | | | |
| ZNF512B | | | | | 20 | | | | | | | | | | |
| MAFB | | | | | 8 | | | | | | | | | | |
| DEK | | | | | | 5 | | | | 14 | | | | | |
| SNAI2 | | | | | | 8 | | | | | | | | 13 | |
| TDG | | | | | | 2 | | | | | | | | 18 | |
| BASP1 | | | | | | 6 | | | 20 | | | | | | |
| ZNF280C | | | | | | 19 | | | | 10 | | | | | |
| TSHZ2 | | | | | | | 17 | | | | | | | | |
| LMX1B | | | | | | | | 15 | | | | | | | |
| SMARCD3 | | | | | | | | 17 | | | | | | | |
| RAD9A | | | | | | | | | 14 | | | | | | |
| DBF4 | | | | | | | | | | 20 | | | | | |
| RBMS1 | | | | | | | | | | 11 | | | | | |
| TRIM32 | | | | | | | | | | | 20 | | | | |
| MEOX2 | | | | | | | | | | | | 7 | | | |
| SP100 | | | | | | | | | | | | 3 | | | |
| HDAC2 | | | | | | | | | | | | | 3 | | |
| RAN | | | | | | | | | | | | | 6 | | |
| SOX11 | | | | | | | | | | | | | 20 | | |
| ZNF697 | | | | | | | | | | | | | | 12 | |
| SNAI1 | | | | | | | | | | | | | | | 10 |
| PKNOX2 | | | | | | | | | | | | | | | 9 |
| HOXA11 | 19 | | | | | | | | | | | | | | |
| ZIC2 | 1 | | | | | | | | | | | | | | |
| PITX1 | 6 | | | | | | | | | | | | | | |
| PSMC3IP | 18 | | | | | | | | | | | | | | |
| HOXC11 | 8 | | | | | | | | | | | | | | |
| SNAPC4 | 13 | | | | | | | | | | | | | | |
| PRMT5 | | 4 | | | | | | | | | | | | | |
| RCOR1 | | 3 | | | | | | | | | | | | | |
| TEAD4 | | 16 | | | | | | | | | | | | | |
| WWTR1 | | 5 | | | | | | | | | | | | | |
| BARX2 | | 15 | | | | | | | | | | | | | |
| CALU | | 6 | | | | | | | | | | | | | |
| CD109 | | 12 | | | | | | | | | | | | | |
| NFIC | | 9 | | | | | | | | | | | | | |
| SOX7 | | 13 | | | | | | | | | | | | | |
| TCF4 | | 17 | | | | | | | | | | | | | |
| ZHX3 | | 8 | | | | | | | | | | | | | |
| PDE3A | | | 15 | | | | | | | | | | | | |
| CCNT1 | | | 20 | | | | | | | | | | | | |
| CLOCK | | | 2 | | | | | | | | | | | | |
| KIAA0754 | | | 16 | | | | | | | | | | | | |
| NCOA2 | | | 8 | | | | | | | | | | | | |
| TAF13 | | | 11 | | | | | | | | | | | | |
| AFF4 | | | 3 | | | | | | | | | | | | |
| MED13 | | | 7 | | | | | | | | | | | | |
| MED23 | | | 17 | | | | | | | | | | | | |
| MTDH | | | 6 | | | | | | | | | | | | |
| PGK1 | | | 4 | | | | | | | | | | | | |
| SMAD5 | | | 18 | | | | | | | | | | | | |
| STON1_GTF2A1L | | | 5 | | | | | | | | | | | | |
| XRCC4 | | | 19 | | | | | | | | | | | | |
| YWHAB | | | 9 | | | | | | | | | | | | |
| ZFHX3 | | | 14 | | | | | | | | | | | | |
| ZNF623 | | | 13 | | | | | | | | | | | | |
| ATF2 | | | | 10 | | | | | | | | | | | |
| ITGB1 | | | | 5 | | | | | | | | | | | |
| PDIA6 | | | | 13 | | | | | | | | | | | |
| TUBB3 | | | | 14 | | | | | | | | | | | |
| ELK3 | | | | 3 | | | | | | | | | | | |
| FNDC3B | | | | 15 | | | | | | | | | | | |
| ITGA5 | | | | 7 | | | | | | | | | | | |
| KIRREL | | | | 20 | | | | | | | | | | | |
| SPRY4 | | | | 6 | | | | | | | | | | | |
| FNDC3A | | | | 11 | | | | | | | | | | | |
| HSP90AB1 | | | | 18 | | | | | | | | | | | |
| KLF7 | | | | 16 | | | | | | | | | | | |
| PEAR1 | | | | 12 | | | | | | | | | | | |
| ZNF281 | | | | 2 | | | | | | | | | | | |
| GLI3 | | | | | 4 | | | | | | | | | | |
| GLIS2 | | | | | 17 | | | | | | | | | | |
| ZEB1 | | | | | 3 | | | | | | | | | | |
| MECP2 | | | | | 14 | | | | | | | | | | |
| HLX | | | | | 1 | | | | | | | | | | |
| MEIS1 | | | | | 5 | | | | | | | | | | |
| ZNF154 | | | | | 13 | | | | | | | | | | |
| ZNF676 | | | | | 11 | | | | | | | | | | |
| HEY1 | | | | | | 4 | | | | | | | | | |
| YAF2 | | | | | | 10 | | | | | | | | | |
| HSF2 | | | | | | 20 | | | | | | | | | |
| TAF9B | | | | | | 7 | | | | | | | | | |
| MAF | | | | | | 1 | | | | | | | | | |
| TP63 | | | | | | 17 | | | | | | | | | |
| AEBP2 | | | | | | 11 | | | | | | | | | |
| DMTF1 | | | | | | 3 | | | | | | | | | |
| HSA_MIR_30E | | | | | | 9 | | | | | | | | | |
| HSA_MIR_3653 | | | | | | 18 | | | | | | | | | |
| MICAL2 | | | | | | | 15 | | | | | | | | |
| RELB | | | | | | | 19 | | | | | | | | |
| C9ORF64 | | | | | | | 16 | | | | | | | | |
| EVC2 | | | | | | | 14 | | | | | | | | |
| CD300E | | | | | | | 18 | | | | | | | | |
| PLEKHN1 | | | | | | | 11 | | | | | | | | |
| BCL3 | | | | | | | 9 | | | | | | | | |
| BHLHE40 | | | | | | | 5 | | | | | | | | |
| EPS8L2 | | | | | | | 20 | | | | | | | | |
| LRRFIP1 | | | | | | | 7 | | | | | | | | |
| DDN | | | | | | | | 5 | | | | | | | |
| FHL2 | | | | | | | | 8 | | | | | | | |
| NFE2L1 | | | | | | | | 4 | | | | | | | |
| ZFP42 | | | | | | | | 10 | | | | | | | |
| POLR2C | | | | | | | | 16 | | | | | | | |
| HOXA1 | | | | | | | | 6 | | | | | | | |
| MSX2 | | | | | | | | 12 | | | | | | | |
| PCGF2 | | | | | | | | 20 | | | | | | | |
| SMYD1 | | | | | | | | 7 | | | | | | | |
| CCND1 | | | | | | | | 1 | | | | | | | |
| E2F4 | | | | | | | | 14 | | | | | | | |
| LHX1 | | | | | | | | 11 | | | | | | | |
| MLXIPL | | | | | | | | 13 | | | | | | | |
| PERINEURAL_INVASION | | | | | | | | 2 | | | | | | | |
| DLX4 | | | | | | | | | 10 | | | | | | |
| ETV6 | | | | | | | | | 6 | | | | | | |
| LBX2 | | | | | | | | | 19 | | | | | | |
| STAT2 | | | | | | | | | 1 | | | | | | |
| ZGLP1 | | | | | | | | | 18 | | | | | | |
| KAT2A | | | | | | | | | 2 | | | | | | |
| IFI16 | | | | | | | | | 11 | | | | | | |
| RUNX1 | | | | | | | | | 12 | | | | | | |
| RBCK1 | | | | | | | | | 17 | | | | | | |
| ZNF335 | | | | | | | | | 13 | | | | | | |
| IRF3 | | | | | | | | | 9 | | | | | | |
| TAF10 | | | | | | | | | 5 | | | | | | |
| TFAP2E | | | | | | | | | 3 | | | | | | |
| ZNF488 | | | | | | | | | | 15 | | | | | |
| AATF | | | | | | | | | | 12 | | | | | |
| PRRX1 | | | | | | | | | | 7 | | | | | |
| AHCTF1 | | | | | | | | | | 13 | | | | | |
| FOXD2 | | | | | | | | | | 6 | | | | | |
| ELF4 | | | | | | | | | | 8 | | | | | |
| HOXA10 | | | | | | | | | | | 14 | | | | |
| SREBF1 | | | | | | | | | | | 8 | | | | |
| HOXA6 | | | | | | | | | | | 16 | | | | |
| PLA2G4A | | | | | | | | | | | 11 | | | | |
| BATF | | | | | | | | | | | 15 | | | | |
| NFKB2 | | | | | | | | | | | 9 | | | | |
| TCF15 | | | | | | | | | | | 17 | | | | |
| LPIN1 | | | | | | | | | | | 19 | | | | |
| STAT6 | | | | | | | | | | | 7 | | | | |
| CC2D1A | | | | | | | | | | | 10 | | | | |
| DAXX | | | | | | | | | | | 3 | | | | |
| ETS2 | | | | | | | | | | | 5 | | | | |
| HOXA7 | | | | | | | | | | | 12 | | | | |
| PPP1R13L | | | | | | | | | | | 13 | | | | |
| TFEB | | | | | | | | | | | 1 | | | | |
| NR2E1 | | | | | | | | | | | | 6 | | | |
| OTP | | | | | | | | | | | | 18 | | | |
| PHTF1 | | | | | | | | | | | | 5 | | | |
| TGIF1 | | | | | | | | | | | | 1 | | | |
| ZNF217 | | | | | | | | | | | | 2 | | | |
| DMRTA2 | | | | | | | | | | | | 8 | | | |
| TEAD3 | | | | | | | | | | | | 11 | | | |
| MYCBP | | | | | | | | | | | | 12 | | | |
| E2F6 | | | | | | | | | | | | | 19 | | |
| HMGA1 | | | | | | | | | | | | | 8 | | |
| PITX2 | | | | | | | | | | | | | 7 | | |
| SMARCD1 | | | | | | | | | | | | | 5 | | |
| YBX1 | | | | | | | | | | | | | 10 | | |
| ZNF207 | | | | | | | | | | | | | 17 | | |
| ENO1 | | | | | | | | | | | | | 9 | | |
| FUBP1 | | | | | | | | | | | | | 13 | | |
| MAFG | | | | | | | | | | | | | 16 | | |
| NPAS2 | | | | | | | | | | | | | | 15 | |
| SMAD3 | | | | | | | | | | | | | | 19 | |
| BCL9L | | | | | | | | | | | | | | 17 | |
| HOXA13 | | | | | | | | | | | | | | 20 | |
| LDB2 | | | | | | | | | | | | | | | 19 |
| ELANE | | | | | | | | | | | | | | | 4 |
| SKI | | | | | | | | | | | | | | | 18 |
| NACC2 | | | | | | | | | | | | | | | 8 |
| TCF21 | | | | | | | | | | | | | | | 1 |
| RARA | | | | | | | | | | | | | | | 11 |
| SMAD7 | | | | | | | | | | | | | | | 3 |
| CALCOCO1 | | | | | | | | | | | | | | | 6 |
| PBX4 | | | | | | | | | | | | | | | 12 |
| SOX18 | | | | | | | | | | | | | | | 20 |
| HNF1B | | | | | | | | | | | | | | | 15 |
| ATOH8 | | | | | | | | | | | | | | | 2 |
| CSRNP1 | | | | | | | | | | | | | | | 14 |

TABLE 3
Top 20 master regulator genes (masters of death), labeled 1-20, for various cancer types. Cancer types corresponding
to the indicated abbreviations are listed in Table 1. n top and top_frequency are described below.

| | Cancer type | | | | | | | | | | | | | | |
|---|---|---|---|---|---|---|---|---|---|---|---|---|---|---|---|
| gene | meso | ov | paad | pcpg | prad | sarc | skcm | stad | tgct | thca | thym | ucec | uvm | n_top | top_frequency |
| MYBL2 | 2 | | | | | | | | | | | 5 | | 6 | 0.21 |
| FOXM1 | | | | | | | 5 | | | | | | | 5 | 0.18 |
| CDK1 | 8 | | | | | | | | | | | | | 5 | 0.18 |
| PTTG1 | 11 | | | | | | | | | | | 15 | | 5 | 0.18 |
| E2F7 | 20 | | 16 | | | | | | | | | | | 5 | 0.18 |
| UHRF1 | 5 | | 17 | | | | | | | | | | | 4 | 0.14 |
| TRIP13 | 3 | | | | | | | | | | | | | 4 | 0.14 |
| AEBP1 | | | | | | | | 4 | | | | 11 | | 4 | 0.14 |
| HDAC7 | | | | | | | | | | | | | | 4 | 0.14 |
| ACTL6A | | | 2 | | | 17 | | | | | | | | 4 | 0.14 |
| ARNTL2 | 4 | | 5 | | | | | | | | | | | 4 | 0.14 |
| TRIM29 | | | 4 | | | | 1 | | | | 13 | | | 4 | 0.14 |
| HMGA2 | | | | | | 18 | | | | | | | | 3 | 0.11 |
| TCF3 | | | | | | 9 | | | | | | | | 3 | 0.11 |
| LOXL2 | 1 | | | | | | | | | | | | | 3 | 0.11 |
| MEIS3 | 9 | | | | | | | 16 | | | | | | 3 | 0.11 |
| TGFB1I1 | 6 | | | | | | | 1 | | | | | | 3 | 0.11 |
| HIC1 | | | | | | | | 3 | | | | | | 3 | 0.11 |
| SPI1 | | | | | | | | 11 | | | | | | 3 | 0.11 |
| FOSL1 | | | | | | | | | | | | | | 3 | 0.11 |
| MMP14 | | | | | | | | | 17 | | | | | 3 | 0.11 |
| VDR | | | | | | | | | | | | | | 3 | 0.11 |
| MAFK | | | | | | | | | | | | | | 3 | 0.11 |
| SLC2A4RG | | | | | | | | | | | | 13 | | 3 | 0.11 |
| NPM1 | | | | | | | | | | | | | | 3 | 0.11 |
| CCNE1 | | | | | | | | | | | | 8 | | 3 | 0.11 |
| CDK2 | | | | | | | 2 | | | | | | | 3 | 0.11 |
| HTATIP2 | | | | | | | | | | | | | 2 | 3 | 0.11 |
| NFE2L3 | | | 10 | | | | | | | | 14 | | | 3 | 0.11 |
| PLSCR1 | | | | | | | | | | | 17 | | | 3 | 0.11 |
| KDM1A | | 13 | | | | 1 | | | | | | | | 3 | 0.11 |
| GRHL2 | | | 1 | | | | 19 | | | | 15 | | | 3 | 0.11 |
| FOXD1 | | | | | | | | | | | | | 14 | 2 | 0.07 |
| EZH2 | 18 | | | | | | | | | | | | | 2 | 0.07 |
| PLK4 | | | | | | | | | | | | | | 2 | 0.07 |
| DNMT1 | 19 | | | | | | | | | | | | | 2 | 0.07 |
| ETV4 | | | | | | | | | | | | | | 2 | 0.07 |
| PCGF6 | | | | | | | | | | | | | | 2 | 0.07 |
| PPRC1 | | | | | | | 20 | | | | | | | 2 | 0.07 |
| ATF6 | | | | 14 | | | | | | | | | | 2 | 0.07 |
| HEYL | | | | | | | | 17 | | | | | | 2 | 0.07 |
| OTX1 | | | | | | | | | | | | | | 2 | 0.07 |
| SSRP1 | | | | | | | | | | | | | | 2 | 0.07 |
| BNC1 | | 14 | | | | | | | | | | | | 2 | 0.07 |
| ZNF521 | | | | | | | | | | | | | | 2 | 0.07 |
| ZNF532 | | | | | | | | | | | | | | 2 | 0.07 |
| REST | | | | | | | | | | | | | | 2 | 0.07 |
| KLF17 | | | | | | | | | 13 | | | | | 2 | 0.07 |
| LIF | | | | | | | | | | | | | 12 | 2 | 0.07 |
| NCOR2 | | | | | | | | | | | | | | 2 | 0.07 |
| SALL2 | | | | | | | | 15 | | | | | | 2 | 0.07 |
| HAND2 | | | | | | | | 7 | | | | | | 2 | 0.07 |
| LZTS1 | | | | | | | | | | | | | | 2 | 0.07 |
| TCF7L1 | | 15 | | | | | | | | | | | | 2 | 0.07 |
| TSHZ3 | | | | | | | | 2 | | | | | | 2 | 0.07 |
| ZNF512B | | | | | | | | | | 11 | | | | 2 | 0.07 |
| MAFB | | | | | | | | 6 | | | | | | 2 | 0.07 |
| DEK | | | | | | | | | | | | | | 2 | 0.07 |
| SNAI2 | | | | | | | | | | | | | | 2 | 0.07 |
| TDG | | | | | | | | | | | | | | 2 | 0.07 |
| BASP1 | | | | | | | | | | | | | | 2 | 0.07 |
| ZNF280C | | | | | | | | | | | | | | 2 | 0.07 |
| TSHZ2 | | | | | | | | | | 4 | | | | 2 | 0.07 |
| LMX1B | | | | | | | | | | 3 | | | | 2 | 0.07 |
| SMARCD3 | | | | | | | | 18 | | | | | | 2 | 0.07 |
| RAD9A | | | | | | | | | | | | 16 | | 2 | 0.07 |
| DBF4 | | | | | 5 | | | | | | | | | 2 | 0.07 |
| RBMS1 | | | 13 | | | | | | | | | | | 2 | 0.07 |
| TRIM32 | | | | | | | 8 | | | | | | | 2 | 0.07 |
| MEOX2 | | | | | | | | 20 | | | | | | 2 | 0.07 |
| SP100 | | | 12 | | | | | | | | | | | 2 | 0.07 |
| HDAC2 | | | | | | 10 | | | | | | | | 2 | 0.07 |
| RAN | | | | | | 13 | | | | | | | | 2 | 0.07 |
| SOX11 | | | | | | 6 | | | | | | | | 2 | 0.07 |
| ZNF697 | | | | | | 8 | | | | | | | | 2 | 0.07 |
| SNAI1 | | | | | | | | | | 5 | | | | 2 | 0.07 |
| PKNOX2 | | | | | | | | 12 | | | | | | 2 | 0.07 |
| E2F1 | 14 | | | | | | | | | | | 4 | | 2 | 0.07 |
| E2F8 | 13 | | 11 | | | | | | | | | | | 2 | 0.07 |
| EHF | | | | 13 | | | | | | | 16 | | | 2 | 0.07 |
| NOC2L | | | | | | 12 | | | | | | 9 | | 2 | 0.07 |
| ZBTB9 | | | | | | 11 | 16 | | | | | | | 2 | 0.07 |
| POU3F1 | | | | | | | 13 | | 18 | | | | | 2 | 0.07 |
| FOSL2 | | | | | | | 18 | | | | 9 | | | 2 | 0.07 |
| FLU | | | | | | | | 13 | | | | | 6 | 2 | 0.07 |
| HOXA11 | | | | | | | | | | | | | | 1 | 0.04 |
| ZIC2 | | | | | | | | | | | | | | 1 | 0.04 |
| PITX1 | | | | | | | | | | | | | | 1 | 0.04 |
| PSMC3IP | | | | | | | | | | | | | | 1 | 0.04 |
| HOXC11 | | | | | | | | | | | | | | 1 | 0.04 |
| SNAPC4 | | | | | | | | | | | | | | 1 | 0.04 |
| PRMT5 | | | | | | | | | | | | | | 1 | 0.04 |
| RCOR1 | | | | | | | | | | | | | | 1 | 0.04 |
| TEAD4 | | | | | | | | | | | | | | 1 | 0.04 |
| WWTR1 | | | | | | | | | | | | | | 1 | 0.04 |
| BARX2 | | | | | | | | | | | | | | 1 | 0.04 |
| CALU | | | | | | | | | | | | | | 1 | 0.04 |
| CD109 | | | | | | | | | | | | | | 1 | 0.04 |
| NFIC | | | | | | | | | | | | | | 1 | 0.04 |
| SOX7 | | | | | | | | | | | | | | 1 | 0.04 |
| TCF4 | | | | | | | | | | | | | | 1 | 0.04 |
| ZHX3 | | | | | | | | | | | | | | 1 | 0.04 |
| PDE3A | | | | | | | | | | | | | | 1 | 0.04 |
| CCNT1 | | | | | | | | | | | | | | 1 | 0.04 |
| CLOCK | | | | | | | | | | | | | | 1 | 0.04 |
| KIAA0754 | | | | | | | | | | | | | | 1 | 0.04 |
| NCOA2 | | | | | | | | | | | | | | 1 | 0.04 |
| TAF13 | | | | | | | | | | | | | | 1 | 0.04 |
| AFF4 | | | | | | | | | | | | | | 1 | 0.04 |
| MED13 | | | | | | | | | | | | | | 1 | 0.04 |
| MED23 | | | | | | | | | | | | | | 1 | 0.04 |
| MTDH | | | | | | | | | | | | | | 1 | 0.04 |
| PGK1 | | | | | | | | | | | | | | 1 | 0.04 |
| SMAD5 | | | | | | | | | | | | | | 1 | 0.04 |
| STON1_GTF2A1L | | | | | | | | | | | | | | 1 | 0.04 |
| XRCC4 | | | | | | | | | | | | | | 1 | 0.04 |
| YWHAB | | | | | | | | | | | | | | 1 | 0.04 |
| ZFHX3 | | | | | | | | | | | | | | 1 | 0.04 |
| ZNF623 | | | | | | | | | | | | | | 1 | 0.04 |
| ATF2 | | | | | | | | | | | | | | 1 | 0.04 |
| ITGB1 | | | | | | | | | | | | | | 1 | 0.04 |
| PDIA6 | | | | | | | | | | | | | | 1 | 0.04 |
| TUBB3 | | | | | | | | | | | | | | 1 | 0.04 |
| ELK3 | | | | | | | | | | | | | | 1 | 0.04 |
| FNDC3B | | | | | | | | | | | | | | 1 | 0.04 |
| ITGA5 | | | | | | | | | | | | | | 1 | 0.04 |
| KIRREL | | | | | | | | | | | | | | 1 | 0.04 |
| SPRY4 | | | | | | | | | | | | | | 1 | 0.04 |
| FNDC3A | | | | | | | | | | | | | | 1 | 0.04 |
| HSP90AB1 | | | | | | | | | | | | | | 1 | 0.04 |
| KLF7 | | | | | | | | | | | | | | 1 | 0.04 |
| PEAR1 | | | | | | | | | | | | | | 1 | 0.04 |
| ZNF281 | | | | | | | | | | | | | | 1 | 0.04 |
| GLI3 | | | | | | | | | | | | | | 1 | 0.04 |
| GLIS2 | | | | | | | | | | | | | | 1 | 0.04 |
| ZEB1 | | | | | | | | | | | | | | 1 | 0.04 |
| MECP2 | | | | | | | | | | | | | | 1 | 0.04 |
| HLX | | | | | | | | | | | | | | 1 | 0.04 |
| MEIS1 | | | | | | | | | | | | | | 1 | 0.04 |
| ZNF154 | | | | | | | | | | | | | | 1 | 0.04 |
| ZNF676 | | | | | | | | | | | | | | 1 | 0.04 |
| HEY1 | | | | | | | | | | | | | | 1 | 0.04 |
| YAF2 | | | | | | | | | | | | | | 1 | 0.04 |
| HSF2 | | | | | | | | | | | | | | 1 | 0.04 |
| TAF9B | | | | | | | | | | | | | | 1 | 0.04 |
| MAF | | | | | | | | | | | | | | 1 | 0.04 |
| TP63 | | | | | | | | | | | | | | 1 | 0.04 |
| AEBP2 | | | | | | | | | | | | | | 1 | 0.04 |
| DMTF1 | | | | | | | | | | | | | | 1 | 0.04 |
| HSA_MIR_30E | | | | | | | | | | | | | | 1 | 0.04 |
| HSA_MIR_3653 | | | | | | | | | | | | | | 1 | 0.04 |
| MICAL2 | | | | | | | | | | | | | | 1 | 0.04 |
| RELB | | | | | | | | | | | | | | 1 | 0.04 |
| C9ORF64 | | | | | | | | | | | | | | 1 | 0.04 |
| EVC2 | | | | | | | | | | | | | | 1 | 0.04 |
| CD300E | | | | | | | | | | | | | | 1 | 0.04 |
| PLEKHN1 | | | | | | | | | | | | | | 1 | 0.04 |
| BCL3 | | | | | | | | | | | | | | 1 | 0.04 |
| BHLHE40 | | | | | | | | | | | | | | 1 | 0.04 |
| EPS8L2 | | | | | | | | | | | | | | 1 | 0.04 |
| LRRFIP1 | | | | | | | | | | | | | | 1 | 0.04 |
| DDN | | | | | | | | | | | | | | 1 | 0.04 |
| FHL2 | | | | | | | | | | | | | | 1 | 0.04 |
| NFE2L1 | | | | | | | | | | | | | | 1 | 0.04 |
| ZFP42 | | | | | | | | | | | | | | 1 | 0.04 |
| POLR2C | | | | | | | | | | | | | | 1 | 0.04 |
| HOXA1 | | | | | | | | | | | | | | 1 | 0.04 |
| MSX2 | | | | | | | | | | | | | | 1 | 0.04 |
| PCGF2 | | | | | | | | | | | | | | 1 | 0.04 |
| SMYD1 | | | | | | | | | | | | | | 1 | 0.04 |
| CCND1 | | | | | | | | | | | | | | 1 | 0.04 |
| E2F4 | | | | | | | | | | | | | | 1 | 0.04 |
| LHX1 | | | | | | | | | | | | | | 1 | 0.04 |
| MLXIPL | | | | | | | | | | | | | | 1 | 0.04 |
| PERINEURAL_INVASION | | | | | | | | | | | | | | 1 | 0.04 |
| DLX4 | | | | | | | | | | | | | | 1 | 0.04 |
| ETV6 | | | | | | | | | | | | | | 1 | 0.04 |
| LBX2 | | | | | | | | | | | | | | 1 | 0.04 |
| STAT2 | | | | | | | | | | | | | | 1 | 0.04 |
| ZGLP1 | | | | | | | | | | | | | | 1 | 0.04 |
| KAT2A | | | | | | | | | | | | | | 1 | 0.04 |
| IFI16 | | | | | | | | | | | | | | 1 | 0.04 |
| RUNX1 | | | | | | | | | | | | | | 1 | 0.04 |
| RBCK1 | | | | | | | | | | | | | | 1 | 0.04 |
| ZNF335 | | | | | | | | | | | | | | 1 | 0.04 |
| IRF3 | | | | | | | | | | | | | | 1 | 0.04 |
| TAF10 | | | | | | | | | | | | | | 1 | 0.04 |
| TFAP2E | | | | | | | | | | | | | | 1 | 0.04 |
| ZNF488 | | | | | | | | | | | | | | 1 | 0.04 |
| AATF | | | | | | | | | | | | | | 1 | 0.04 |
| PRRX1 | | | | | | | | | | | | | | 1 | 0.04 |
| AHCTF1 | | | | | | | | | | | | | | 1 | 0.04 |
| FOXD2 | | | | | | | | | | | | | | 1 | 0.04 |
| ELF4 | | | | | | | | | | | | | | 1 | 0.04 |
| HOXA10 | | | | | | | | | | | | | | 1 | 0.04 |
| SREBF1 | | | | | | | | | | | | | | 1 | 0.04 |
| HOXA6 | | | | | | | | | | | | | | 1 | 0.04 |
| PLA2G4A | | | | | | | | | | | | | | 1 | 0.04 |
| BATF | | | | | | | | | | | | | | 1 | 0.04 |
| NFKB2 | | | | | | | | | | | | | | 1 | 0.04 |
| TCF15 | | | | | | | | | | | | | | 1 | 0.04 |
| LPIN1 | | | | | | | | | | | | | | 1 | 0.04 |
| STAT6 | | | | | | | | | | | | | | 1 | 0.04 |
| CC2D1A | | | | | | | | | | | | | | 1 | 0.04 |
| DAXX | | | | | | | | | | | | | | 1 | 0.04 |
| ETS2 | | | | | | | | | | | | | | 1 | 0.04 |
| HOXA7 | | | | | | | | | | | | | | 1 | 0.04 |
| PPP1R13L | | | | | | | | | | | | | | 1 | 0.04 |
| TFEB | | | | | | | | | | | | | | 1 | 0.04 |
| NR2E1 | | | | | | | | | | | | | | 1 | 0.04 |
| OTP | | | | | | | | | | | | | | 1 | 0.04 |
| PHTF1 | | | | | | | | | | | | | | 1 | 0.04 |
| TGIF1 | | | | | | | | | | | | | | 1 | 0.04 |
| ZNF217 | | | | | | | | | | | | | | 1 | 0.04 |
| DMRTA2 | | | | | | | | | | | | | | 1 | 0.04 |
| TEAD3 | | | | | | | | | | | | | | 1 | 0.04 |
| MYCBP | | | | | | | | | | | | | | 1 | 0.04 |
| E2F6 | | | | | | | | | | | | | | 1 | 0.04 |
| HMGA1 | | | | | | | | | | | | | | 1 | 0.04 |
| PITX2 | | | | | | | | | | | | | | 1 | 0.04 |
| SMARCD1 | | | | | | | | | | | | | | 1 | 0.04 |
| YBX1 | | | | | | | | | | | | | | 1 | 0.04 |
| ZNF207 | | | | | | | | | | | | | | 1 | 0.04 |
| ENO1 | | | | | | | | | | | | | | 1 | 0.04 |
| FUBP1 | | | | | | | | | | | | | | 1 | 0.04 |
| MAFG | | | | | | | | | | | | | | 1 | 0.04 |
| NPAS2 | | | | | | | | | | | | | | 1 | 0.04 |
| SMAD3 | | | | | | | | | | | | | | 1 | 0.04 |
| BCL9L | | | | | | | | | | | | | | 1 | 0.04 |
| HOXA13 | | | | | | | | | | | | | | 1 | 0.04 |
| LDB2 | | | | | | | | | | | | | | 1 | 0.04 |
| ELANE | | | | | | | | | | | | | | 1 | 0.04 |
| SKI | | | | | | | | | | | | | | 1 | 0.04 |
| NACC2 | | | | | | | | | | | | | | 1 | 0.04 |
| TCF21 | | | | | | | | | | | | | | 1 | 0.04 |
| RARA | | | | | | | | | | | | | | 1 | 0.04 |
| SMAD7 | | | | | | | | | | | | | | 1 | 0.04 |
| CALCOCO1 | | | | | | | | | | | | | | 1 | 0.04 |
| PBX4 | | | | | | | | | | | | | | 1 | 0.04 |
| SOX18 | | | | | | | | | | | | | | 1 | 0.04 |
| HNF1B | | | | | | | | | | | | | | 1 | 0.04 |
| ATOH8 | | | | | | | | | | | | | | 1 | 0.04 |
| CSRNP1 | | | | | | | | | | | | | | 1 | 0.04 |
| BRCA1 | 10 | | | | | | | | | | | | | 1 | 0.04 |
| BRIP1 | 15 | | | | | | | | | | | | | 1 | 0.04 |
| DNMT3B | 7 | | | | | | | | | | | | | 1 | 0.04 |
| MYBL1 | 12 | | | | | | | | | | | | | 1 | 0.04 |
| BEND6 | 16 | | | | | | | | | | | | | 1 | 0.04 |
| NRG1 | 17 | | | | | | | | | | | | | 1 | 0.04 |
| ZNF90 | | 16 | | | | | | | | | | | | 1 | 0.04 |
| HCG22 | | 9 | | | | | | | | | | | | 1 | 0.04 |
| ARID1B | | 19 | | | | | | | | | | | | 1 | 0.04 |
| TEX261 | | 7 | | | | | | | | | | | | 1 | 0.04 |
| SLC1A6 | | 8 | | | | | | | | | | | | 1 | 0.04 |
| SOCS5 | | 6 | | | | | | | | | | | | 1 | 0.04 |
| ZNF781 | | 12 | | | | | | | | | | | | 1 | 0.04 |
| HTR3C | | 5 | | | | | | | | | | | | 1 | 0.04 |
| PAX3 | | 17 | | | | | | | | | | | | 1 | 0.04 |
| STAC2 | | 4 | | | | | | | | | | | | 1 | 0.04 |
| BUD31 | | 20 | | | | | | | | | | | | 1 | 0.04 |
| NFKBIB | | 1 | | | | | | | | | | | | 1 | 0.04 |
| CDSN | | 10 | | | | | | | | | | | | 1 | 0.04 |
| HIF3A | | 18 | | | | | | | | | | | | 1 | 0.04 |
| PER1 | | 3 | | | | | | | | | | | | 1 | 0.04 |
| PFDN5 | | 11 | | | | | | | | | | | | 1 | 0.04 |
| SNORD15A | | 2 | | | | | | | | | | | | 1 | 0.04 |
| KLF5 | | | 6 | | | | | | | | | | | 1 | 0.04 |
| POU2F3 | | | 18 | | | | | | | | | | | 1 | 0.04 |
| PTPN14 | | | 20 | | | | | | | | | | | 1 | 0.04 |
| YAP1 | | | 3 | | | | | | | | | | | 1 | 0.04 |
| MSLN | | | 15 | | | | | | | | | | | 1 | 0.04 |
| KLF3 | | | 14 | | | | | | | | | | | 1 | 0.04 |
| AHR | | | 8 | | | | | | | | | | | 1 | 0.04 |
| ZFP36L1 | | | 7 | | | | | | | | | | | 1 | 0.04 |
| NMI | | | 9 | | | | | | | | | | | 1 | 0.04 |
| YY1 | | | 19 | | | | | | | | | | | 1 | 0.04 |
| BRCA2 | | | | 2 | | | | | | | | | | 1 | 0.04 |
| CASC5 | | | | 18 | | | | | | | | | | 1 | 0.04 |
| COPA | | | | 8 | | | | | | | | | | 1 | 0.04 |
| LHX4 | | | | 3 | | | | | | | | | | 1 | 0.04 |
| RFX5 | | | | 6 | | | | | | | | | | 1 | 0.04 |
| ZBTB37 | | | | 4 | | | | | | | | | | 1 | 0.04 |
| BLZF1 | | | | 5 | | | | | | | | | | 1 | 0.04 |
| C11ORF42 | | | | 1 | | | | | | | | | | 1 | 0.04 |
| IRF6 | | | | 7 | | | | | | | | | | 1 | 0.04 |
| TAF2 | | | | 20 | | | | | | | | | | 1 | 0.04 |
| ZNF157 | | | | 15 | | | | | | | | | | 1 | 0.04 |
| ZNF195 | | | | 12 | | | | | | | | | | 1 | 0.04 |
| S100A5 | | | | 10 | | | | | | | | | | 1 | 0.04 |
| TTTY14 | | | | 9 | | | | | | | | | | 1 | 0.04 |
| TSG101 | | | | 19 | | | | | | | | | | 1 | 0.04 |
| PAX5 | | | | 17 | | | | | | | | | | 1 | 0.04 |
| TFAP2B | | | | 11 | | | | | | | | | | 1 | 0.04 |
| PATE2 | | | | 16 | | | | | | | | | | 1 | 0.04 |
| CIZ1 | | | | | 18 | | | | | | | | | 1 | 0.04 |
| NUP62 | | | | | 6 | | | | | | | | | 1 | 0.04 |
| POLE3 | | | | | 4 | | | | | | | | | 1 | 0.04 |
| POP1 | | | | | 17 | | | | | | | | | 1 | 0.04 |
| RAB14 | | | | | 16 | | | | | | | | | 1 | 0.04 |
| TIAL1 | | | | | 14 | | | | | | | | | 1 | 0.04 |
| KIAA0319 | | | | | 8 | | | | | | | | | 1 | 0.04 |
| QTRTD1 | | | | | 12 | | | | | | | | | 1 | 0.04 |
| ZNF57 | | | | | 10 | | | | | | | | | 1 | 0.04 |
| MBD1 | | | | | 1 | | | | | | | | | 1 | 0.04 |
| U2AF2 | | | | | 2 | | | | | | | | | 1 | 0.04 |
| GAS2 | | | | | 3 | | | | | | | | | 1 | 0.04 |
| KCNC3 | | | | | 13 | | | | | | | | | 1 | 0.04 |
| NCBP2 | | | | | 11 | | | | | | | | | 1 | 0.04 |
| DDX27 | | | | | 20 | | | | | | | | | 1 | 0.04 |
| SLC12A5 | | | | | 19 | | | | | | | | | 1 | 0.04 |
| GGA3 | | | | | 9 | | | | | | | | | 1 | 0.04 |
| SRC | | | | | 15 | | | | | | | | | 1 | 0.04 |
| ZNF274 | | | | | 7 | | | | | | | | | 1 | 0.04 |
| GMEB1 | | | | | | 7 | | | | | | | | 1 | 0.04 |
| MEX3A | | | | | | 14 | | | | | | | | 1 | 0.04 |
| SERBP1 | | | | | | 15 | | | | | | | | 1 | 0.04 |
| TARDBP | | | | | | 2 | | | | | | | | 1 | 0.04 |
| LHX8 | | | | | | 5 | | | | | | | | 1 | 0.04 |
| MYBBP1A | | | | | | 16 | | | | | | | | 1 | 0.04 |
| MAGED1 | | | | | | 19 | | | | | | | | 1 | 0.04 |
| C1QBP | | | | | | 20 | | | | | | | | 1 | 0.04 |
| HES6 | | | | | | | 9 | | | | | | | 1 | 0.04 |
| MED15 | | | | | | | 14 | | | | | | | 1 | 0.04 |
| OVOL1 | | | | | | | 7 | | | | | | | 1 | 0.04 |
| PA2G4 | | | | | | | 15 | | | | | | | 1 | 0.04 |
| GATAD2A | | | | | | | 10 | | | | | | | 1 | 0.04 |
| SOX15 | | | | | | | 17 | | | | | | | 1 | 0.04 |
| TFAP2A | | | | | | | 6 | | | | | | | 1 | 0.04 |
| ZNF750 | | | | | | | 11 | | | | | | | 1 | 0.04 |
| SLC38A8 | | | | | | | 12 | | | | | | | 1 | 0.04 |
| OVOL2 | | | | | | | 4 | | | | | | | 1 | 0.04 |
| ERG | | | | | | | | 10 | | | | | | 1 | 0.04 |
| PTGER3 | | | | | | | | 19 | | | | | | 1 | 0.04 |
| RUNX1T1 | | | | | | | | 8 | | | | | | 1 | 0.04 |
| ZFPM2 | | | | | | | | 5 | | | | | | 1 | 0.04 |
| FOXC2 | | | | | | | | | 12 | | | | | 1 | 0.04 |
| FOXD3 | | | | | | | | | 9 | | | | | 1 | 0.04 |
| HOXD11 | | | | | | | | | 4 | | | | | 1 | 0.04 |
| LIMS3 | | | | | | | | | 11 | | | | | 1 | 0.04 |
| TREX2 | | | | | | | | | 10 | | | | | 1 | 0.04 |
| ZSCAN10 | | | | | | | | | 6 | | | | | 1 | 0.04 |
| HSA_MIR_483 | | | | | | | | | 2 | | | | | 1 | 0.04 |
| IGF2 | | | | | | | | | 3 | | | | | 1 | 0.04 |
| SOX2 | | | | | | | | | 19 | | | | | 1 | 0.04 |
| TNFRSF1A | | | | | | | | | 16 | | | | | 1 | 0.04 |
| TFE3 | | | | | | | | | 20 | | | | | 1 | 0.04 |
| ZFP57 | | | | | | | | | 7 | | | | | 1 | 0.04 |
| CDX4 | | | | | | | | | 14 | | | | | 1 | 0.04 |
| DPPA2 | | | | | | | | | 15 | | | | | 1 | 0.04 |
| LOC100287704 | | | | | | | | | 8 | | | | | 1 | 0.04 |
| ZNF679 | | | | | | | | | 5 | | | | | 1 | 0.04 |
| ANTXR1 | | | | | | | | | | 19 | | | | 1 | 0.04 |
| DCAF17 | | | | | | | | | | 10 | | | | 1 | 0.04 |
| SIX2 | | | | | | | | | | 12 | | | | 1 | 0.04 |
| UCHL5 | | | | | | | | | | 16 | | | | 1 | 0.04 |
| PIAS2 | | | | | | | | | | 1 | | | | 1 | 0.04 |
| SMAD1 | | | | | | | | | | 13 | | | | 1 | 0.04 |
| ZFHX4 | | | | | | | | | | 18 | | | | 1 | 0.04 |
| PEG3 | | | | | | | | | | 8 | | | | 1 | 0.04 |
| SMAD9 | | | | | | | | | | 9 | | | | 1 | 0.04 |
| GZF1 | | | | | | | | | | 6 | | | | 1 | 0.04 |
| ZFP41 | | | | | | | | | | 17 | | | | 1 | 0.04 |
| SIX4 | | | | | | | | | | 15 | | | | 1 | 0.04 |
| MED13L | | | | | | | | | | 20 | | | | 1 | 0.04 |
| NR0B2 | | | | | | | | | | 14 | | | | 1 | 0.04 |
| PPARGC1A | | | | | | | | | | 2 | | | | 1 | 0.04 |
| PRDM12 | | | | | | | | | | 7 | | | | 1 | 0.04 |
| ZNF462 | | | | | | | | | | | 20 | | | 1 | 0.04 |
| FXN | | | | | | | | | | | 6 | | | 1 | 0.04 |
| JUN | | | | | | | | | | | 19 | | | 1 | 0.04 |
| HDAC9 | | | | | | | | | | | 5 | | | 1 | 0.04 |
| PBX3 | | | | | | | | | | | 3 | | | 1 | 0.04 |
| LPIN3 | | | | | | | | | | | 1 | | | 1 | 0.04 |
| ZNF80 | | | | | | | | | | | 7 | | | 1 | 0.04 |
| EOMES | | | | | | | | | | | 10 | | | 1 | 0.04 |
| BATF2 | | | | | | | | | | | 11 | | | 1 | 0.04 |
| CIITA | | | | | | | | | | | 18 | | | 1 | 0.04 |
| PRDM1 | | | | | | | | | | | 8 | | | 1 | 0.04 |
| ZBTB7B | | | | | | | | | | | 2 | | | 1 | 0.04 |
| ZNF768 | | | | | | | | | | | 12 | | | 1 | 0.04 |
| SPIC | | | | | | | | | | | 4 | | | 1 | 0.04 |
| FOXN4 | | | | | | | | | | | | 19 | | 1 | 0.04 |
| MED8 | | | | | | | | | | | | 12 | | 1 | 0.04 |
| TRIB3 | | | | | | | | | | | | 7 | | 1 | 0.04 |
| DDX41 | | | | | | | | | | | | 1 | | 1 | 0.04 |
| HGS | | | | | | | | | | | | 3 | | 1 | 0.04 |
| DRAP1 | | | | | | | | | | | | 20 | | 1 | 0.04 |
| CCDC137 | | | | | | | | | | | | 2 | | 1 | 0.04 |
| GMEB2 | | | | | | | | | | | | 18 | | 1 | 0.04 |
| RFX2 | | | | | | | | | | | | 17 | | 1 | 0.04 |
| THRB | | | | | | | | | | | | 6 | | 1 | 0.04 |
| DMAP1 | | | | | | | | | | | | 14 | | 1 | 0.04 |
| RBPJL | | | | | | | | | | | | 10 | | 1 | 0.04 |
| GLI2 | | | | | | | | | | | | | 5 | 1 | 0.04 |
| TSC22D1 | | | | | | | | | | | | | 13 | 1 | 0.04 |
| GATA6 | | | | | | | | | | | | | 3 | 1 | 0.04 |
| GLIS3 | | | | | | | | | | | | | 8 | 1 | 0.04 |
| FOXF1 | | | | | | | | | | | | | 18 | 1 | 0.04 |
| NR5A2 | | | | | | | | | | | | | 1 | 1 | 0.04 |
| BATF3 | | | | | | | | | | | | | 7 | 1 | 0.04 |
| IRF1 | | | | | | | | | | | | | 10 | 1 | 0.04 |
| SNCAIP | | | | | | | | | | | | | 16 | 1 | 0.04 |
| CITED1 | | | | | | | | | | | | | 4 | 1 | 0.04 |
| CEBPG | | | | | | | | | | | | | 19 | 1 | 0.04 |
| IRF5 | | | | | | | | | | | | | 15 | 1 | 0.04 |
| BCL11B | | | | | | | | | | | | | 11 | 1 | 0.04 |
| XBP1 | | | | | | | | | | | | | 17 | 1 | 0.04 |
| ZNF576 | | | | | | | | | | | | | 20 | 1 | 0.04 |
| SAP30 | | | | | | | | | | | | | 9 | 1 | 0.04 |

n_top is the number of cancer subtypes out of 28 cancer subtypes for which the indicated gene is considered a top master regulator of poor prognosis, i.e., top 20 highest rank for this tumor. top_frequency is the percentage of n_top/total number of cancer subtypes (28). A high n_top and top_frequency indicates that this gene is a master regulator of poor prognosis across many cancer subtypes which is an indication that this gene plays an important role in patient deaths.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### Example 2: Cancer Diagnosis

Analysis of master regulator genes can be used in the diagnosis of cancer. Diagnosis can be used to detect cancer, monitor cancer, detect risk of developing cancer, or analyze prognosis in a patient known to have cancer.

A sample is collected from a patient having cancer, suspected of having cancer, or suspected of being at risk of developing cancer and expression of at least one gene of a master regulator in the sample is measured. The expression of one or more mater regulator genes in the patient sample is then compared with expression the same master regulator genes in a corresponding healthy reference sample. Increased expression in the patient sample relative to the healthy reference sample is an indicator of the possible presence of cancer, risk of developing cancer, or of poor prognosis.

An increase in the expression of one or more of MYBL2, PTTG1, FOXM1, E2F7, CDK1, UHRF1, TRIP13, TRIM29, HDAC7, ARNTL2, AEBP1, or ACTL6A relative to the healthy reference sample indicates the possible presence of cancer, risk of developing cancer, or poor prognosis in a patient with cancer.

An increase in the expression of one or more of ARNTL2, LOXL2, FOXM1, MAFK, MMP14, TRIM29, FOSL1, CDK1, E2F7, ZNF697, SNAI2, PLSCR1, NPAS2, PLK4, BCL9L, TDG, SMAD3, HOXA13, MYBL2, or BRIP1 relative to the healthy reference sample indicates the possible presence of lung adenocarcinoma, risk of developing lung adenocarcinoma, or poor prognosis in a patient known to have lung adenocarcinoma.

An increase in the expression of one or more of TCF21, ATOH8, SMAD7, ELANE, NCOR2, CALCOCO1, HIC1, NACC2, PKNOX2, SNAIL RARA, PBX4, MAFK, CSRNP1, HNF1B, SPI1, HDAC7, SKI LDB2, or SOX18 relative to the healthy reference sample indicates the possible presence of lung squamous cell carcinoma, risk of developing lung squamous cell carcinoma, or poor prognosis in a patient known to have lung squamous cell carcinoma.

An increase in the expression of one or more of CLOCK, AFF4, PGK1, STON1_GTF2A1L, MTDH, MED13, NCOA2, YWHAB, TAF13, REST, ZNF623, ZFHX3, PDE3A, KIAA0754, MED23, SMAD5, XRCC4, CCNT1, ADAMTS12, or ZNF699 relative to the healthy reference sample indicates the possible presence of breast invasive carcinoma, risk of developing breast invasive carcinoma, or poor prognosis in a patient known to have breast invasive carcinoma.

An increase in the expression of one or more of MBD1, U2AF2, GAS2, POLE3, DBF4, NUP62, ZNF274, KIAA0319, GGA3, ZNF57, NCBP2, QTRTD1, KCNC3, TIAL1, SRC, RAB14, POP1, CIZ1, SLC12A5, or DDX27 relative to the healthy reference sample indicates the possible presence of prostate adenocarcinoma, risk of developing prostate adenocarcinoma, or poor prognosis in a patient known to have prostate adenocarcinoma.

An increase in the expression of one or more of HLX, AEBP1, ZEB1, GLI3, MEIS1, MEIS3, TCF7L1, MAFB, TSHZ3, TGFB1I1, ZNF676, HAND2, ZNF154, MECP2, ZNF521, HDAC7, GLIS2, LZTS1, HIC1, or ZNF512B relative to the healthy reference sample indicates the possible presence of colon and/or rectum adenocarcinoma, risk of developing colon and/or rectum adenocarcinoma, or poor prognosis in a patient known to have colon and/or rectum adenocarcinoma.

An increase in the expression of one or more of GRHL2, ACTL6A, YAP1, TRIM29, ARNTL2, KLF5, ZFP36L1, AHR, NMI, NFE2L3, E2F8, SP100, RBMS1, KLF3, MSLN, E2F7, UHRF1, POU2F3, YY1, or PTPN14 relative to the healthy reference sample indicates the possible presence of pancreatic adenocarcinoma, risk of developing pancreatic adenocarcinoma, or poor prognosis in a patient known to have pancreatic adenocarcinoma.

An increase in the expression of one or more of TRIP13, MYBL2, HDAC2, PTTG1, SMARCD1, RAN, PITX2, HMGA1, ENO1, YBX1, NPM1, CDK1, FUBP1, ACTL6A, SSRP1, MAFG, ZNF207, KDM1A, E2F6, or SOX11 relative to the healthy reference sample indicates the possible presence of liver hepatocellular carcinoma, risk of developing liver hepatocellular carcinoma, or poor prognosis in a patient known to have liver hepatocellular carcinoma.

An increase in the expression of one or more of TFEB, VDR, DAXX, HTATIP2, ETS2, HDAC7, STAT6, SREBF1, NFKB2, CC2D1A, PLA2G4A, HOXA7, PPP1R13L, HOXA10, BATF, HOXA6, TCF15, ZNF532, LPIN1, or TRIM32 relative to the healthy reference sample indicates the possible presence of acute myeloid leukemia, risk of developing acute myeloid leukemia, or poor prognosis in a patient known to have acute myeloid leukemia.

An increase in the expression of one or more of NFKBIB, SNORD15A, PER1, STAC2, HTR3C, SOCS5, TEX261, SLC1A6, HCG22, CDSN, PFDN5, ZNF781, KDM1A, BNC1, TCF7L1, ZNF90, PAX3, HIF3A, ARID1B, or BUD31 relative to the healthy reference sample indicates the possible presence of ovarian serous cystadenocarcinoma, risk of developing ovarian serous cystadenocarcinoma, or poor prognosis in a patient known to have ovarian serous cystadenocarcinoma.

An increase in the expression of one or more of VDR, MMP14, HDAC7, AEBP1, BHLHE40, FOSL1, LRRFIP1, LZTS1, BCL3, SLC2A4RG, PLEKHN1, MAFK, ETV4, EVC2, MICAL2, C9ORF64, TSHZ2, CD300E, RELB, or EPS8L2 relative to the healthy reference sample indicates the possible presence of glioblastoma multiforme, risk of developing glioblastoma multiforme, or poor prognosis in a patient known to have glioblastoma multiforme.

Similarly, an increase in the expression of one or more of the genes labeled 1-20 in any one of the columns in Tables 2 and 3 relative to expression of the same gene(s) in the healthy reference sample indicates the possible presence, risk, or poor prognosis for the cancer type listed at the top of the corresponding column.

In some embodiments, a statistically significant increase in expression of at least one master regulator indicates a poor prognosis, reduced response to standard treatment or a predicted decrease in survival time.

In some embodiments, a statistically significant increase in expression of at least two master regulators indicates a poor prognosis, reduced response to standard treatment or a predicted reduced survival time.

In some embodiments, a statistically significant increase in expression of at least three master regulators indicates a poor prognosis, reduced response to standard treatment or a predicted reduced survival time.

### Example 3: Predicting Patient Survival

Calculating Risk Scores: Risk scores are calculated by comparing the expression level of one or more master regulator genes involved in certain pathways in a test sample with the expression levels of the same genes in a healthy reference sample. Increased expression in the test sample relative to the healthy reference sample is an indicator of increased risk. A positive risk score indicates increased expression of one or more master regulator genes in a pathway of A, B, C, or D (below) of FIG. 17 in a test sample from a subject relative to expression of the same gene(s) in a healthy reference sample.

A) Cell cycle risk score is determined by obtaining a risk score for one or more master regulators selected from the group consisting of: CDK2, CCNE1, FOXM1, UHRFI, CDK1, PTTG1, MYBL2, and TRIP13.

B) Epigenetic/chromosome remodeling risk score is determined by obtaining a risk score for one or more master regulators selected from the group consisting of: RAN, ACTL6A, NPMI, HDAC2, SOX11, KDM1A, NOC2L, ZBTB9, ZNF697, TRIM32, PPRC1, POU3F1, BNC1, ATF6, OTX1, SSRP1, ETV4, EZH2, DNMT1, PLK4, E2F8, E2F1, DBF4, RAD9A, ZNF280C, DEK, PCGF6, and TDG

C) Epithelial Mesenchymal Transitions (EMT) risk score is determined by obtaining a risk score for one or more master regulators selected from the group consisting of: SNAI2, E2F7, ARNTL2, LOXL2, HMGA4, MMP14, FOSL1, LIF, FOXD1, LMX1B, TSHZ2, ZNF512B, SNAIL MEOX2, C2A4RG, MAFK, NCOR2, ZNF532, HADC7, VDR, HTATIP2, NFE2L3, SP100, REST, PLSCR1, FOSL2, TRIM29, and GRHL2.

D) Immune/development risk score is determined by obtaining a risk score for one or more master regulators selected from the group consisting of: EHF, RBMS1, FLI1, MAFB, SPI1, BASP1, SMARCD3, HAND2, TCFL1, TSHZ3, ZNF521, HEYL, PKNOX2, HIC1, SALL2, KLF17, MEIS3, TGFB1I1, LZTS1, and AEBP1.

Prognosis and/or prediction of patient survival can be analyzed across multiple cancer type by analyzing expression of master regulators in various pathways, including epithelial mesenchymal transition (EMT), cell cycle, angiogenesis, immune response, and inflammatory response.

A sample is collected from a patient having cancer, suspected of having cancer, or at risk of developing cancer and expression of at least one gene of a master regulator in the sample is measured. The expression of the master regulator gene in the patient sample is then compared with expression of the master regulator gene in a corresponding healthy reference sample. Increased expression of the master regulator in the patient sample relative to the healthy reference sample is an indicator of poor prognosis or reduced predicted survival time.

In some embodiments, master regulator gene is in the hallmark epithelial mesenchymal transition pathway, reactome cell cycle pathway, angiogenesis pathway, immune response pathway, or inflammatory response pathway.

Master regulator genes in the EMT hallmark pathway can be selected from the group consisting of: ZNF469, PRRX1, AEBP1, MEIS3, SNAIL MMP14, ADAMTS12, ITGA5, TGFB1I1, and CREB3L1.

Master regulator genes in the reactome cell cycle pathway can be selected from the group consisting of: MYBL2, CDK1, TRIP13, EZH2, FOXM1, UHRF1, PTTG1, E2F7, BRCA1, and E2F8.

Master regulator genes in the angiogenesis pathway can be selected from the group consisting of: HEYL, LZTS1, COL4A1, ERG, SOX18, LDB2, GJC1, HLX, SOX17, and PDE3A.

Master regulator genes in the immune response pathway can be selected from the group consisting of: SPI1, IRF1, GATA3, IL2RB, BCL3, FOXP3, ACAP1, GBP1, CXCL13, and WWTR1.

Master regulator genes in the inflammatory response pathway can be selected from the group consisting of: SPI1, MS4A4A, CIITA, MAFB, VDR, BCL3, LILRB2, IRF5, WWTR1, and CALU.

### Example 5: Master of Death Gene VDR

VDR is ranked Pt in Glioblastoma multiforme (GBM) and surprisingly 2^{nd} in Acute Myeloid Leukemia (AML, termed LAML in Tables 1 and 2). AML is a very phenotypically distinct cancer from GBM. Further, these two very different cancers share three common master of death genes: VDR, HDAC7 and NFKB2/RELB (Table 4). The identification of common masters of death indicate these two cancers are more similar molecularly than previous physiological and/or morphological data would suggest.

TABLE 4
Masters of Death in Glioblastoma multiforme
Acute Myeloid Leukemia.

| Master | Glioblastoma | Master | Acute Myeloid |
|---|---|---|---|
| of death | multiforme | of death | Leukemia |
| VDR | 1 | TFEB | 1 |
| MMP14 | 2 | VDR | 2 |
| HDAC7 | 3 | DAXX | 3 |
| AEBP1 | 4 | HTATIP2 | 4 |
| BHLHE40 | 5 | ETS2 | 5 |
| FOSL1 | 6 | HDAC7 | 6 |
| LRRFIP1 | 7 | STAT6 | 7 |
| LZTS1 | 8 | SREBF1 | 8 |
| BCL3 | 9 | NFKB2 | 9 |
| SLC2A4RG | 10 | CC2D1A | 10 |
| PLEKHN1 | 11 | PLA2G4A | 11 |
| MAFK | 12 | HOXA7 | 12 |
| ETV4 | 13 | PPP1R13L | 13 |
| EVC2 | 14 | HOXA10 | 14 |
| MICAL2 | 15 | BATF | 15 |
| C9ORF64 | 16 | HOXA6 | 16 |
| TSHZ2 | 17 | TCF15 | 17 |
| CD300E | 18 | ZNF532 | 18 |
| RELB | 19 | LPIN1 | 19 |
| EPS8L2 | 20 | TRIM32 | 20 |

VDR-dependent regulation of immune activation (i.e. innate immune) is well characterized. However, a role for VDR in regulating cancer stem cell factors (e.g., SOX2, BHLHE40, SNAI2) was not previously recognized. Many of top 50 genes downstream and connected with VDR are immune related due to VDR's role in innate immunity. Without wishing to be bound by theory, is it possible the regulation of immune activation could be the mechanism of how VDR regulates poor prognosis and survival, i.e. by regulating cancer stem cells, which are the cells that can propagate cancers, are resistant to therapy, and a major cause of tumor recurrence and poor prognosis.

### Gene Networks

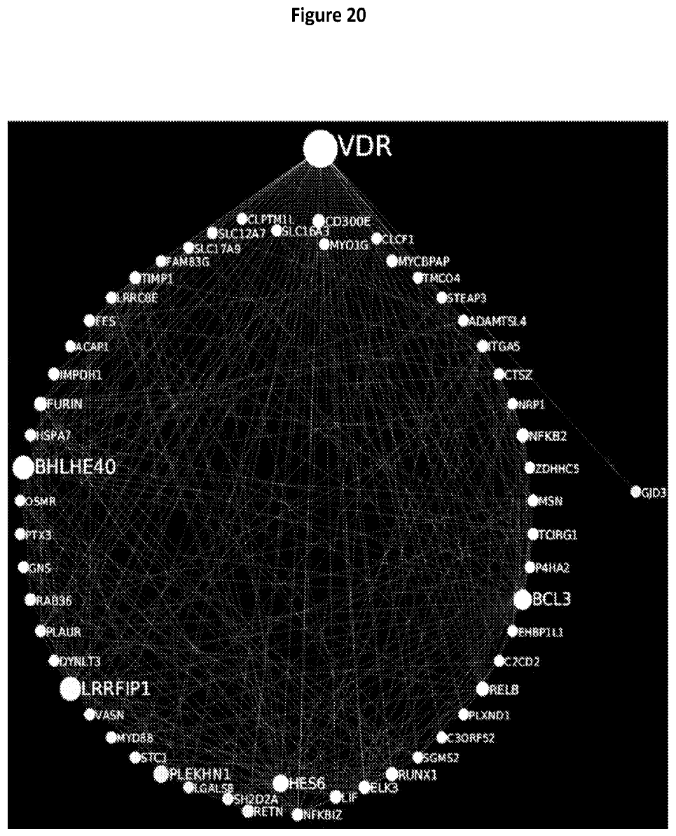

Gene network analysis using GeneRep/nSCORE indicates the VDR is networked with BHLHE40, BCL3, NFKB2, RELB, LRRFIP1, HES6, among others (Table 5 and FIG. 20).

Gene network analysis using GeneRep/nSCORE indicates that BHLHE40, TCF12, BCL3, SOX2, and RELP are networked with genes up-regulated in response to low oxygen levels (hypoxia); genes defining the EMT, as in wound healing, fibrosis and metastasis; genes up-regulated through activation of mTORC1 complex; heparin binding; and response to hypoxia.

Gene network analysis using GeneRep/nSCORE indicates that VDR, ZFP1, MYO1C, NFKB2, and ARID3A are networked with GTPAse activity, RNA polymerase II core promoter proximal region sequence-specific DNA binding; membrane raft; pathways of cancer, and multicellular organism development.

Gene network analysis using GeneRep/nSCORE indicates that SNAI2, CYGB, ZIC3, SEMA3F, and LAMB1 are networked with angiogenesis; extracellular matrix organization; proteinaceous extracellular matrix; positive regulation of cell proliferation; and focal adhesion.

Gene network analysis using GeneRep/nSCORE indicates that VDR1, BHLHE40, TCF12, BCL3, and SOX2 are networked with genes involved in immune system; inflammatory response; neutrophil degranulation; immune response; and innate immune response.

TABLE 5
GBM Network of top VDR connected genes.

| | | |
|---|---|---|
| CLCF1 | 23529 | Interleukin-6 family signaling |
| | | Cytokine Signaling in Immune system |
| MYCBPAP | 84073 | May play a role in spermatogenesis |
| STEAP3 | 55240 | DNA Damage Response |
| | | Direct p53 effectors |
| ADAMTSL4 | 54507 | O-glycosylation of TSR domain-containing |
| | | proteins |
| | | O-linked glycosylation |
| ITGA5 | 3678 | Developmental Biology |
| | | Shigellosis |
| CTSZ | 1522 | Lysosome |
| | | Innate Immune System |
| NRP1 | 8829 | Developmental Biology |
| | | Apoptotic Pathways in Synovial Fibroblasts |
| NFKB2 | 4791 | TNFR1 Pathway |
| | | Interleukin-1 processing |
| ZDHHC5 | 25921 | |
| MSN | 4478 | RhoA signaling pathway |
| | | Diseases associated with MSN include |
| | | Immunodeficiency 50 and Verrucous carcinoma |
| TCIRG1 | 10312 | Insulin receptor recycling |
| | | Lysosome |
| P4HA2 | 8974 | Collagen chain trimerization |
| | | Metabolism |
| BCL3 | 602 | Apoptosis-related network due to altered |
| | | Notch3 in ovarian cancer |
| | | NF-KappaB Family Pathway |
| RELB | 5971 | TNFR1 Pathway |
| | | CD209 (DC-SIGN) signaling |
| PLXND1 | 23129 | Semaphorin interactions |
| SGMS2 | 166929 | Metabolism and Sphingolipid metabolism |
| RUNX1 | 861 | Transport of glucose and other sugars, |
| | | bile salts and organic acids, metal |
| | | ions and amine compounds |
| | | Transcriptional misregulation in cancer |
| ELK3 | 2004 | ID signaling pathway |
| | | ERK Signaling |
| LIF | 3976 | PEDF Induced Signaling |
| | | PAK Pathway |
| | | Diseases associated with LIF include |
| | | Leukemia and Ectopic Pregnancy |
| NFKBIZ | 64332 | Transcriptional misregulation in cancer |
| | | NF-kappaB Signaling |
| HES6 | 55502 | Notch signaling pathway (KEGG |
| | | Notch-mediated HES/HEY network |

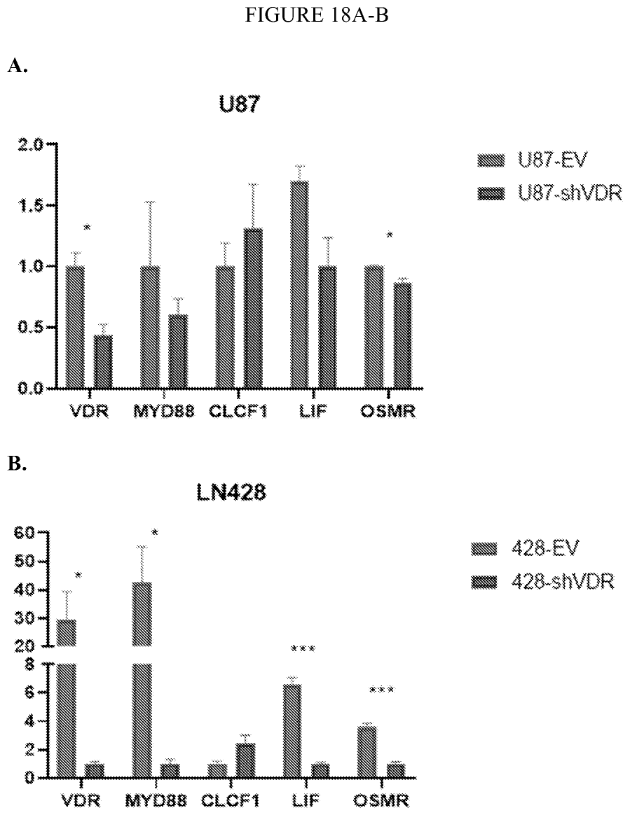

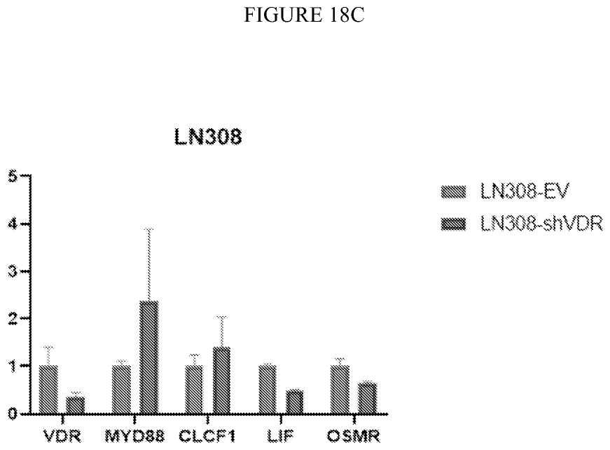

We show that VDR knockdown is correlated with downregulation of two key downstream immune factors, LIF and OSMR, in three GBM cell lines examined. These data confirm that VDR is linked to immune regulation (FIGS. 18A-B).

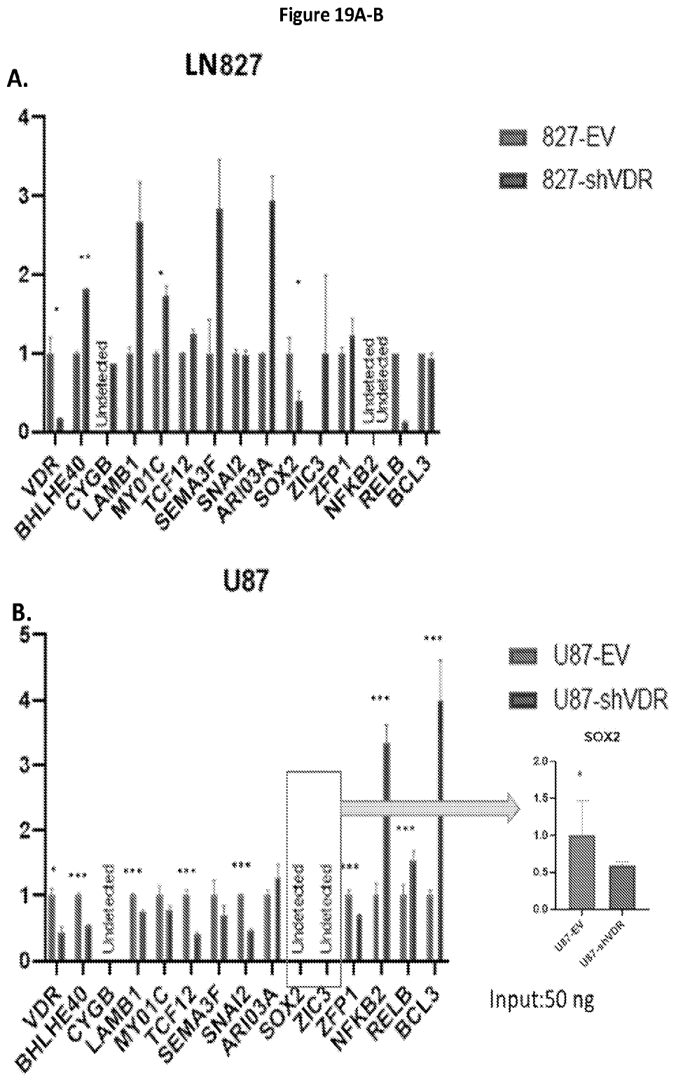

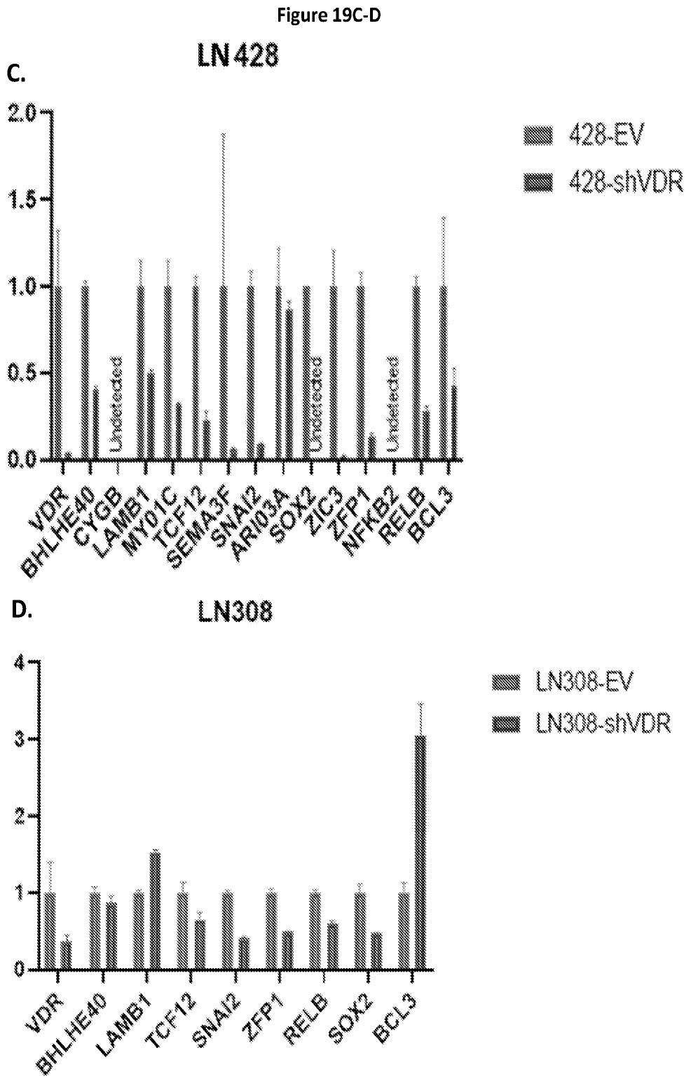

In four independent human GBM cell lines, VDR was shown to be required for the expression of Sox2 (FIGS. 19A-B). In U87 cells, we had to increase the input RNA from 10 to 50 ng in order to detect Sox2 and showed that the correlation between VDR and Sox2 also held true in this cell. Other cancer stem cell factors, BHLHE40, SNAI2 and ZFP1 are also regulated by VDR in three out of the four cell lines. This finding confirms the connection between VDR and cancer stem cell factors, especially Sox2, which was not previously known.

### Example 6: Role of VDR and Sox2 in GBM Prognosis

Analysis of whether VDR knockdown impairs GSC viability and whether Sox2 expression can rescue VDR knockdown effect is performed to examine the role of the VDR and Sox2 link in GBM stem cells formation and survival.

The influence of vitamin D is also examined for its role the link between VDR (vitamin D receptor) and GBM prognosis. Addition of vitamin D is used to determine if vitamin D will direct VDR to a beneficial pro-immune function of liganded VDR. Removal of vitamin D is used to determine if vitamin D deficiency leads to harmful pro-tumor unliganded VDR signal.

We show that the identified masters of death regulate critical pathways that have been shown to be critical for aggressive phenotypes and treatment resistance in cancers.

Further we show that the masters of death are shared among cancers of a similar cellular origin or type, that cancers can be classified into distinct groups based on their master of death profiles even is the cancers are not traditionally thought of a phenotypically similar, and that targeting these shared common masters of death have a potential to impact efficacy in multiple cancers.

The validation of the above masters of death in distinct cancer types, demonstrates that predictive quality of the method if identifying target genes for diagnosis and/or treatment of cancer.

### Example 7: Master of Death Genes MYBL2, FOXM1, and PTTG1

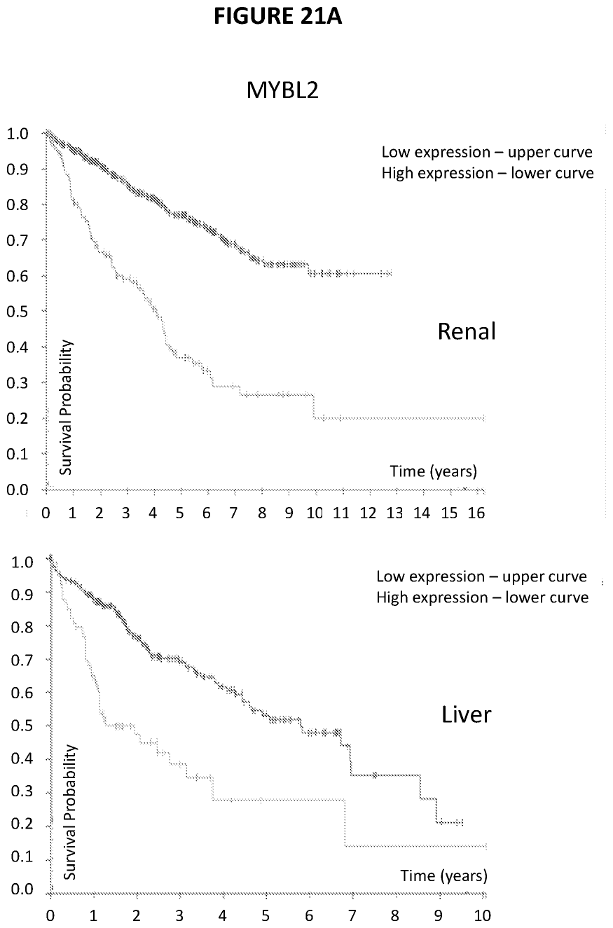

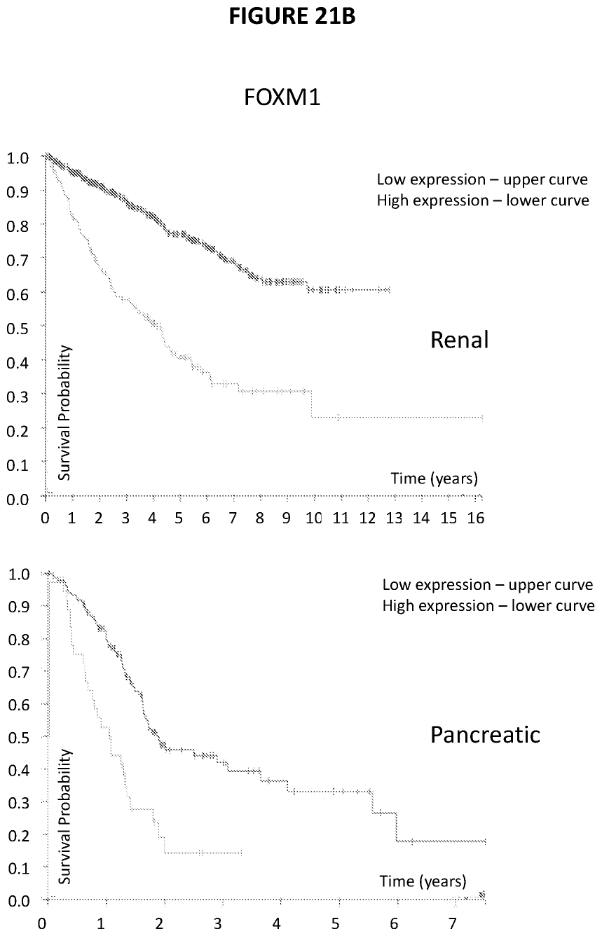

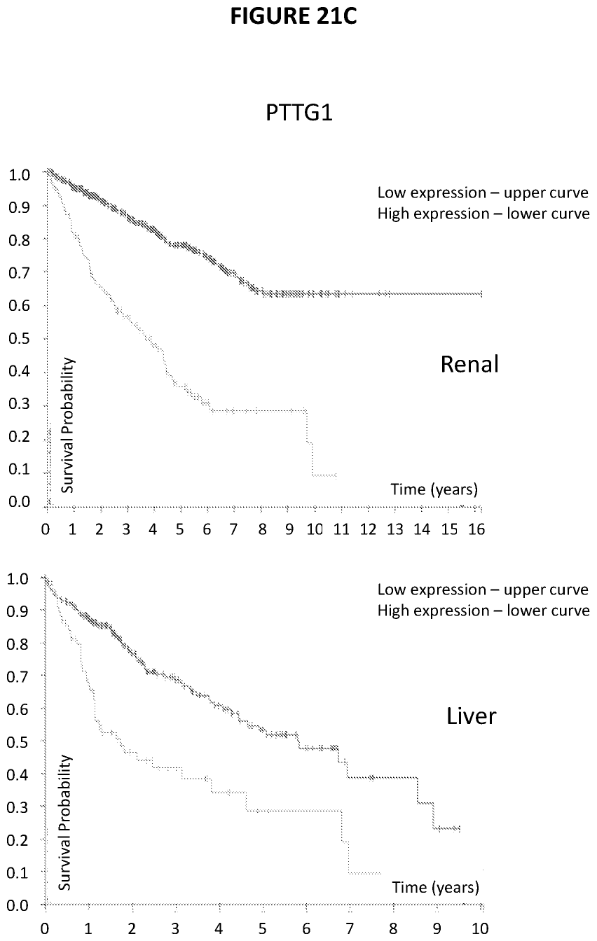

MYBL2, FOXM1, and PTTG1 are shared and correlated with poor survival in more than multiple cancer types. We have shown that MYBL2 is a master regulator in renal and liver cancer; FOXM1 is a master regulator in renal and pancreatic cancer, and PTTG1 is a master regulator in renal and liver cancer. For each of these genes, high expression correlated with decreased survival probability (FIG. 21A-C). The identification of common master regulators indicates these cancers are more similar molecularly than previous physiological and/or morphological data would suggest.

### Example 8: Treating Cancer

The cancers listed in Tables 2 and 3 can be treated by administering immunotherapy compositions, small molecules, RNA interference agents, antisense oligonucleotides, or combinations thereof that target one or more of the master regulators associated with the cancer.

What is claimed is:

1. A method for detecting poor prognosis in a subject with cancer comprising measuring the expression of MYBL2 in a sample from the subject with cancer and comparing the expression with a healthy reference sample, wherein increased expression in the sample from the subject with cancer relative to the healthy reference sample is an indicator of poor prognosis.

2. A method for detecting poor prognosis in a subject with cancer comprising measuring the expression of one or more of MYBL2, PTTG1, FOXM1, E2F7, and CDK1 in a sample from the subject with cancer and comparing the expression with a healthy reference sample, wherein increased expression in the sample from the subject with cancer relative to the healthy reference sample is an indicator of poor prognosis.

3. A method for detecting poor prognosis in a subject with cancer comprising measuring the expression of one or more of UHRF1, TRIP13, TRIM29, HDAC7, ARNTL2, AEBP1, or ACTL6A in a sample from the subject with cancer and comparing the expression with a healthy reference sample, wherein increased expression in the sample from the subject with cancer relative to the healthy reference sample is an indicator of poor prognosis.

4. A method for detecting poor prognosis in a subject with lung adenocarcinoma comprising measuring the expression of ARNTL2 in a sample from the subject and comparing the expression with a healthy reference sample, wherein increased expression in the sample from the subject relative to the healthy reference sample is an indicator of poor prognosis.

5. The method of claim 4, further comprising measuring the expression of at least one of LOXL2, FOXM1, MAFK, MMP14, TRIM29, FOSL1, CDK1, E2F7, ZNF697, SNAI2, PLSCR1, NPAS2, PLK4, BCL9L, TDG, SMAD3, HOXA13, MYBL2, or BRIP1 in the sample from the subject and comparing the expression with a healthy reference sample, wherein increased expression in the sample from the subject relative to the healthy reference sample is an indicator of poor prognosis.

6. A method for detecting poor prognosis in a subject with lung squamous cell carcinoma comprising measuring the expression of TCF21 in a sample from the subject and comparing the expression with a healthy reference sample, wherein increased expression in the sample from the subject relative to the healthy reference sample is an indicator of poor prognosis.

7. The method of claim 6, further comprising measuring the expression of at least one of ATOH8, SMAD7, ELANE, NCOR2, CALCOCO1, HIC1, NACC2, PKNOX2, SNAI1, RARA, PBX4, MAFK, CSRNP1, HNF1B, SPI1, HDAC7, SKI LDB2, or SOX18 in the sample from the subject and comparing the expression with a healthy reference sample, wherein increased expression in the sample from the subject relative to the healthy reference sample is an indicator of poor prognosis.

8. A method for detecting poor prognosis in a subject with breast invasive carcinoma comprising measuring the expression of CLOCK in a sample from the subject and comparing the expression with a healthy reference sample, wherein increased expression in the sample from the subject relative to the healthy reference sample is an indicator of poor prognosis.

9. The method of claim 8, further comprising measuring the expression of at least one of AFF4, PGK1, STON1_GTF2A1L, MTDH, MED13, NCOA2, YWHAB, TAF13, REST, ZNF623, ZFHX3, PDE3A, KIAA0754, MED23, SMAD5, XRCC4, CCNT1, ADAMTS12, or ZNF699 in the sample from the subject and comparing the expression with a healthy reference sample, wherein increased expression in the sample from the subject relative to the healthy reference sample is an indicator of poor prognosis.

10. A method for detecting poor prognosis in a subject with prostate adenocarcinoma comprising measuring the expression of MBD1 in a sample from the subject and comparing the expression with a healthy reference sample, wherein increased expression in the sample from the subject relative to the healthy reference sample is an indicator of poor prognosis.

11. The method of claim 10, further comprising measuring the expression of at least one of U2AF2, GAS2, POLE3, DBF4, NUP62, ZNF274, KIAA0319, GGA3, ZNF57, NCBP2, QTRTD1, KCNC3, TIAL1, SRC, RAB14, POP1, CIZ1, SLC12A5, or DDX27 in the sample from the subject and comparing the expression with a healthy reference sample, wherein increased expression in the sample from the subject relative to the healthy reference sample is an indicator of poor prognosis.

12. A method for detecting poor prognosis in a subject with colon and/or rectum adenocarcinoma comprising measuring the expression of HLX in a sample from the subject and comparing the expression with a healthy reference sample, wherein increased expression in the sample from the subject relative to the healthy reference sample is an indicator of poor prognosis.

13. The method of claim 10, further comprising measuring the expression of at least one of AEBP1, ZEB1, GLI3, MEIS1, MEIS3, TCF7L1, MAFB, TSHZ3, TGFB1I1, ZNF676, HAND2, ZNF154, MECP2, ZNF521, HDAC7, GLIS2, LZTS1, HIC1, or ZNF512B in the sample from the subject and comparing the expression with a healthy reference sample, wherein increased expression in the sample from the subject relative to the healthy reference sample is an indicator of poor prognosis.

14. A method for detecting poor prognosis in a subject with pancreatic adenocarcinoma comprising measuring the expression of GRHL2 in a sample from the subject and comparing the expression with a healthy reference sample, wherein increased expression in the sample from the subject relative to the healthy reference sample is an indicator of poor prognosis.

15. The method of claim 14, further comprising measuring the expression of at least one of ACTL6A, YAP1, TRIM29, ARNTL2, KLF5, ZFP36L1, AHR, NMI, NFE2L3, E2F8, SP100, RBMS1, KLF3, MSLN, E2F7, UHRF1, POU2F3, YY1, or PTPN14 in the sample from the subject and comparing the expression with a healthy reference sample, wherein increased expression in the sample from the subject relative to the healthy reference sample is an indicator of poor prognosis.

16. A method for detecting poor prognosis in a subject with liver hepatocellular carcinoma comprising measuring the expression of TRIP13 in a sample from the subject and comparing the expression with a healthy reference sample, wherein increased expression in the sample from the subject relative to the healthy reference sample is an indicator of poor prognosis.

17. The method of claim 16, further comprising measuring the expression of at least one of MYBL2, HDAC2, PTTG1, SMARCD1, RAN, PITX2, HMGA1, ENO1, YBX1, NPM1, CDK1, FUBP1, ACTL6A, SSRP1, MAFG, ZNF207, KDM1A, E2F6, or SOX11 in the sample from the subject and comparing the expression with a healthy reference sample, wherein increased expression in the sample from the subject relative to the healthy reference sample is an indicator of poor prognosis.

18. A method for detecting poor prognosis in a subject with acute myeloid leukemia comprising measuring the expression of TFEB in a sample from the subject and comparing the expression with a healthy reference sample, wherein increased expression in the sample from the subject relative to the healthy reference sample is an indicator of poor prognosis.

19. The method of claim 18, further comprising measuring the expression of at least one of VDR, DAXX, HTATIP2, ETS2, HDAC7, STAT6, SREBF1, NFKB2, CC2D1A, PLA2G4A, HOXA7, PPP1R13L, HOXA10, BATF, HOXA6, TCF15, ZNF532, LPIN1, or TRIM32 in the sample from the subject and comparing the expression with a healthy reference sample, wherein increased expression in the sample from the subject relative to the healthy reference sample is an indicator of poor prognosis.

20. A method for detecting poor prognosis in a subject with ovarian serous cystadenocarcinoma comprising measuring the expression of NFKBIB in a sample from the subject and comparing the expression with a healthy reference sample, wherein increased expression in the sample from the subject relative to the healthy reference sample is an indicator of poor prognosis.

21. The method of claim 20, further comprising measuring the expression of at least one of SNORD15A, PER1, STAC2, HTR3C, SOCS5, TEX261, SLC1A6, HCG22, CDSN, PFDN5, ZNF781, KDM1A, BNC1, TCF7L1, ZNF90, PAX3, HIF3A, ARID1B, or BUD31 in the sample from the subject and comparing the expression with a healthy reference sample, wherein increased expression in the sample from the subject relative to the healthy reference sample is an indicator of poor prognosis.

22. A method for detecting poor prognosis in a subject with glioblastoma multiforme comprising measuring the expression of VDR in a sample from the subject and comparing the expression with a reference sample, wherein increased expression in the sample from the subject relative to the healthy reference sample is an indicator of poor prognosis.

23. The method of claim 22, further comprising measuring the expression of at least one of MMP14, HDAC7, AEBP1, BHLHE40, FOSL1, LRRFIP1, LZTS1, BCL3, SLC2A4RG, PLEKHN1, MAFK, ETV4, EVC2, MICAL2, C9ORF64, TSHZ2, CD300E, RELB, or EPS8L2 in the sample from the subject and comparing the expression with a healthy reference sample, wherein increased expression in the sample from the subject relative to the healthy reference sample is an indicator of poor prognosis.

24. A method for predicting patient survival in multiple cancer types comprising measuring in a sample from a cancer patient the expression of at least one of 409 genes for master regulators of poor prognosis which are selected from the group consisting of: MYBL2, FOXM1, CDK1, PTTG1, E2F7, UHRF1, TRIP13, AEBP1, HDAC7, ACTL6A, ARNTL2, TRIM29, HMGA2, TCF3, LOXL2, MEIS3, TGFB1I1, HIC1, SPI1, FOSL1, MMP14, VDR, MAFK, SLC2A4RG, NPM1, CCNE1, CDK2, HTATIP2, NFE2L3, PLSCR1, KDM1A, GRHL2, FOXD1, EZH2, PLK4, DNMT1, ETV4, PCGF6, PPRC1, ATF6, HEYL, OTX1, SSRP1, BNC1, ZNF521, ZNF532, REST, KLF17, LIF, NCOR2, SALL2, HAND2, LZTS1, TCF7L1, TSHZ3, ZNF512B, MAFB, DEK, SNAI2, TDG, BASP1, ZNF280C, TSHZ2, LMX1B, SMARCD3, RAD9A, DBF4, RBMS1, TRIM32, MEOX2, SP100, HDAC2, RAN, SOX11, ZNF697, SNAIL PKNOX2, E2F1, E2F8, EHF, NOC2L, ZBTB9, POU3F1, FOSL2, FLI1, HOXA11, ZIC2, PITX1, PSMC3IP, HOXC11, SNAPC4, PRMT5, RCOR1, TEAD4, WWTR1, BARX2, CALU, CD109, NFIC, SOX7, TCF4, ZHX3, PDE3A, CCNT1, CLOCK, KIAA0754, NCOA2, TAF13, AFF4, MED13, MED23, MTDH, PGK1, SMAD5, STON1_GTF2A1L, XRCC4, YWHAB, ZFHX3, ZNF623, ATF2, ITGB1, PDIA6, TUBB3, ELKS, FNDC3B, ITGA5, KIRREL, SPRY4, FNDC3A, HSP90AB1, KLF7, PEAR1, ZNF281, GLI3, GLIS2, ZEB1, MECP2, HLX, MEIS1, ZNF154, ZNF676, HEY1, YAF2, HSF2, TAF9B, MAF, TP63, AEBP2, DMTF1, HSA_MIR_30E, HSA_MIR_3653, MICAL2, RELB, C9ORF64, EVC2, CD300E, PLEKHN1, BCL3, BHLHE40, EPS8L2, LRRFIP1, DDN, FHL2, NFE2L1, ZFP42, POLR2C, HOXA1, MSX2, PCGF2, SMYD1, CCND1, E2F4, LHX1, MLXIPL, PERINEURAL_INVASION, DLX4, ETV6, LBX2, STAT2, ZGLP1, KAT2A, IFI16, RUNX1, RBCK1, ZNF335, IRF3, TAF10, TFAP2E, ZNF488, AATF, PRRX1, AHCTF1, FOXD2, ELF4, HOXA10, SREBF1, HOXA6, PLA2G4A, BATF, NFKB2, TCF15, LPIN1, STAT6, CC2D1A, DAXX, ETS2, HOXA7, PPP1R13L, TFEB, NR2E1, OTP, PHTF1, TGIF1, ZNF217, DMRTA2, TEAD3, MYCBP, E2F6, HMGA1, PITX2, SMARCD1, YBX1, ZNF207, ENO1, FUBP1, MAFG, NPAS2, SMAD3, BCL9L, HOXA13, LDB2, ELANE, SKI, NACC2, TCF21, RARA, SMAD7, CALCOCO1, PBX4, SOX18, HNF1B, ATOH8, CSRNP1, BRCA1, BRIP1, DNMT3B, MYBL1, BEND6, NRG1, ZNF90, HCG22, ARID1B, TEX261, SLC1A6, SOCS5, ZNF781, HTR3C, PAX3, STAC2, BUD31, NFKBIB, CDSN, HIF3A, PER1, PFDN5, SNORD15A, KLF5, POU2F3, PTPN14, YAP1, MSLN, KLF3, AHR, ZFP36L1, NMI, YY1, BRCA2, CASC5, COPA, LHX4, RFX5, ZBTB37, BLZF1, C11ORF42, IRF6, TAF2, ZNF157, ZNF195, S100A5, TTTY14, TSG101, PAX5, TFAP2B, PATE2, CIZ1, NUP62, POLE3, POP1, RAB14, TIAL1, KIAA0319, QTRTD1, ZNF57, MBD1, U2AF2, GAS2, KCNC3, NCBP2, DDX27, SLC12A5, GGA3, SRC, ZNF274, GMEB1, MEX3A, SERBP1, TARDBP, LHX8, MYBBP1A, MAGED1, C1QBP, HES6, MED15, OVOL1, PA2G4, GATAD2A, SOX15, TFAP2A, ZNF750, SLC38A8, OVOL2, ERG, PTGER3, RUNX1T1, ZFPM2, FOXC2, FOXD3, HOXD11, LIMS3, TREX2, ZSCAN10, HSA_MIR_483, IGF2, SOX2, TNFRSF1A, TFE3, ZFP57, CDX4, DPPA2, LOC100287704, ZNF679, ANTXR1, DCAF17, SIX2, UCHL5, PIAS2, SMAD1, ZFHX4, PEG3, SMAD9, GZF1, ZFP41, SIX4, MED13L, NR0B2, PPARGC1A, PRDM12, ZNF462, FXN, JUN, HDAC9, PBX3, LPIN3, ZNF80, EOMES, BATF2, CIITA, PRDM1, ZBTB7B, ZNF768, SPIC, FOXN4, MEDS, TRIB3, DDX41, HGS, DRAP1, CCDC137, GMEB2, RFX2, THRB, DMAP1, RBPJL, GLI2, TSC22D1, GATA6, GLIS3, FOXF1, NR5A2, BATF3, IRF1, SNCAIP, CITED1, CEBPG, IRF5, BCL11B, XBP1, ZNF576, and SAP30 and comparing the expression with a healthy reference sample, wherein increased expression in the sample from the patient relative to the healthy reference sample is an indicator of reduced predicted survival time.

25. A method for predicting patient survival in multiple cancer types comprising measuring in a sample from a cancer patient the expression of at least one of 85 genes for master regulators of poor prognosis which are selected from the group consisting of: MYBL2, FOXM1, CDK1, PTTG1, E2F7, UHRF1, TRIP13, AEBP1, HDAC7, ACTL6A, ARNTL2, TRIM29, HMGA2, TCF3, LOXL2, MEIS3, TGFB1I1, HIC1, SPI1, FOSL1, MMP14, VDR, MAFK, SLC2A4RG, NPM1, CCNE1, CDK2, HTATIP2, NFE2L3, PLSCR1, KDM1A, GRHL2, FOXD1, EZH2, PLK4, DNMT1, ETV4, PCGF6, PPRC1, ATF6, HEYL, OTX1, SSRP1, BNC1, ZNF521, ZNF532, REST, KLF17, LIF, NCOR2, SALL2, HAND2, LZTS1, TCF7L1, TSHZ3, ZNF512B, MAFB, DEK, SNAI2, TDG, BASP1, ZNF280C, TSHZ2, LMX1B, SMARCD3, RAD9A, DBF4, RBMS1, TRIM32, MEOX2, SP100, HDAC2, RAN, SOX11, ZNF697, SNAIL PKNOX2, E2F1, E2F8, EHF, NOC2L, ZBTB9, POU3F1, FOSL2, and FLI1 and comparing the expression with a healthy reference sample, wherein increased expression in the sample from the patient relative to the healthy reference sample is an indicator of reduced predicted survival time.

26. The method of claim 25, further comprising calculating cell cycle, epigenetic/chromosome remodeling, Epithelial Mesenchymal Transitions (EMT), immune/development risk scores.

27. The method of claim 26, wherein calculating the cell cycle risk score comprises measuring the expression of at least one of CDK2, CCNE1, FOXM1, UHRFI, CDK1, PTTG1, MYBL2, or TRIP13.

28. The method of claim 26, wherein calculating the epigenetic risk score comprises measuring the expression of at least one of RAN, ACTL6A, NPMI, HDAC2, SOX11, KDM1A, NOC2L, ZBTB9, ZNF697, TRIM32, PPRC1, POU3F1, BNC1, ATF6, OTX1, SSRP1, ETV4, EZH2, DNMT1, PLK4, E2F8, E2F1, DBF4, RAD9A, ZNF280C, DEK, PCGF6, or TDG.

29. The method of claim 26, wherein calculating the EMT risk score comprises measuring the expression of at least one of SNAI2, E2F7, ARNTL2, LOXL2, HMGA4, MMP14, FOSL1, LIF, FOXD1, LMX1B, TSHZ2, ZNF512B, SNAI1, MEOX2, C2A4RG, MAFK, NCOR2, ZNF532, HADC7, VDR, HTATIP2, NFE2L3, SP100, REST, PLSCR1, FOSL2, TRIM29, or GRHL2.

30. The method of claim 26, wherein calculating the immune/developmental risk score comprises measuring the expression of at least one of EHF, RBMS1, FLI1, MAFB, SPI1, BASP1, SMARCD3, HAND2, TCFL1, TSHZ3, ZNF521, HEYL, PKNOX2, HIC1, SALL2, KLF17, MEIS3, TGFB1I1, LZTS1, or AEBP1.

31. A method for treating a subject with cancer comprising administering an immunotherapy composition or small molecule that targets a master regulator of poor prognosis.

32. The method of claim 31, wherein the immunotherapy composition comprises a peptide formulation derived from a master regulator of poor prognosis or a nanoparticle or dendritic cell containing peptides derived from a master regulator of poor prognosis.

33. The method of claim 31, wherein the immunotherapy composition comprises a nanoparticle or dendritic cells containing RNA which codes for a master regulator of poor prognosis.

34. The method of any one of claims 31-33, wherein the master regulator of poor prognosis is selected from the group consisting of: MYBL2, FOXM1, CDK1, PTTG1, E2F7, UHRF1, TRIP13, AEBP1, HDAC7, ACTL6A, ARNTL2, TRIM29, HMGA2, TCF3, LOXL2, MEIS3, TGFB1I1, HIC1, SPI1, FOSL1, MMP14, VDR, MAFK, SLC2A4RG, NPM1, CCNE1, CDK2, HTATIP2, NFE2L3, PLSCR1, KDM1A, GRHL2, FOXD1, EZH2, PLK4, DNMT1, ETV4, PCGF6, PPRC1, ATF6, HEYL, OTX1, SSRP1, BNC1, ZNF521, ZNF532, REST, KLF17, LIF, NCOR2, SALL2, HAND2, LZTS1, TCF7L1, TSHZ3, ZNF512B, MAFB, DEK, SNAI2, TDG, BASP1, ZNF280C, TSHZ2, LMX1B, SMARCD3, RAD9A, DBF4, RBMS1, TRIM32, MEOX2, SP100, HDAC2, RAN, SOX11, ZNF697, SNAIL PKNOX2, E2F1, E2F8, EHF, NOC2L, ZBTB9, POU3F1, FOSL2, FLI1, HOXA11, ZIC2, PITX1, PSMC3IP, HOXC11, SNAPC4, PRMT5, RCOR1, TEAD4, WWTR1, BARX2, CALU, CD109, NFIC, SOX7, TCF4, ZHX3, PDE3A, CCNT1, CLOCK, KIAA0754, NCOA2, TAF13, AFF4, MED13, MED23, MTDH, PGK1, SMAD5, STON1_GTF2A1L, XRCC4, YWHAB, ZFHX3, ZNF623, ATF2, ITGB1, PDIA6, TUBB3, ELK3, FNDC3B, ITGA5, KIRREL, SPRY4, FNDC3A, HSP90AB1, KLF7, PEAR1, ZNF281, GLI3, GLIS2, ZEB1, MECP2, HLX, MEIS1, ZNF154, ZNF676, HEY1, YAF2, HSF2, TAF9B, MAF, TP63, AEBP2, DMTF1, HSA_MIR_30E, HSA_MIR_3653, MICAL2, RELB, C9ORF64, EVC2, CD300E, PLEKHN1, BCL3, BHLHE40, EPS8L2, LRRFIP1, DDN, FHL2, NFE2L1, ZFP42, POLR2C, HOXA1, MSX2, PCGF2, SMYD1, CCND1, E2F4, LHX1, MLXIPL, PERINEURAL_INVASION, DLX4, ETV6, LBX2, STAT2, ZGLP1, KAT2A, IFI16, RUNX1, RBCK1, ZNF335, IRF3, TAF10, TFAP2E, ZNF488, AATF, PRRX1, AHCTF1, FOXD2, ELF4, HOXA10, SREBF1, HOXA6, PLA2G4A, BATF, NFKB2, TCF15, LPIN1, STAT6, CC2D1A, DAXX, ETS2, HOXA7, PPP1R13L, TFEB, NR2E1, OTP, PHTF1, TGIF1, ZNF217, DMRTA2, TEAD3, MYCBP, E2F6, HMGA1, PITX2, SMARCD1, YBX1, ZNF207, ENO1, FUBP1, MAFG, NPAS2, SMAD3, BCL9L, HOXA13, LDB2, ELANE, SKI, NACC2, TCF21, RARA, SMAD7, CALCOCO1, PBX4, SOX18, HNF1B, ATOH8, CSRNP1, BRCA1, BRIP1, DNMT3B, MYBL1, BEND6, NRG1, ZNF90, HCG22, ARID1B, TEX261, SLC1A6, SOCS5, ZNF781, HTR3C, PAX3, STAC2, BUD31, NFKBIB, CDSN, HIF3A, PER1, PFDN5, SNORD15A, KLF5, POU2F3, PTPN14, YAP1, MSLN, KLF3, AHR, ZFP36L1, NMI, YY1, BRCA2, CASC5, COPA, LHX4, RFX5, ZBTB37, BLZF1, C11ORF42, IRF6, TAF2, ZNF157, ZNF195, S100A5, TTTY14, TSG101, PAX5, TFAP2B, PATE2, CIZ1, NUP62, POLE3, POP1, RAB14, TIAL1, KIAA0319, QTRTD1, ZNF57, MBD1, U2AF2, GAS2, KCNC3, NCBP2, DDX27, SLC12A5, GGA3, SRC, ZNF274, GMEB1, MEX3A, SERBP1, TARDBP, LHX8, MYBBP1A, MAGED1, C1QBP, HES6, MED15, OVOL1, PA2G4, GATAD2A, SOX15, TFAP2A, ZNF750, SLC38A8, OVOL2, ERG, PTGER3, RUNX1T1, ZFPM2, FOXC2, FOXD3, HOXD11, LIMS3, TREX2, ZSCAN10, HSA_MIR_483, IGF2, SOX2, TNFRSF1A, TFE3, ZFP57, CDX4, DPPA2, LOC100287704, ZNF679, ANTXR1, DCAF17, SIX2, UCHL5, PIAS2, SMAD1, ZFHX4, PEG3, SMAD9, GZF1, ZFP41, SIX4, MED13L, NR0B2, PPARGC1A, PRDM12, ZNF462, FXN, JUN, HDAC9, PBX3, LPIN3, ZNF80, EOMES, BATF2, CIITA, PRDM1, ZBTB7B, ZNF768, SPIC, FOXN4, MEDS, TRIB3, DDX41, HGS, DRAP1, CCDC137, GMEB2, RFX2, THRB, DMAP1, RBPJL, GLI2, TSC22D1, GATA6, GLIS3, FOXF1, NR5A2, BATF3, IRF1, SNCAIP, CITED1, CEBPG, IRF5, BCL11B, XBP1, ZNF576, and SAP30.

35. The method of any one of claims 31-33, wherein the master regulator of poor prognosis is selected from the group consisting of: CDK2, CCNE1, FOXM1, UHRFI, CDK1, PTTG1, MYBL2, and TRIP13.

36. The method of any one of claims 31-33, wherein the master regulator of poor prognosis is selected from the group consisting of: RAN, ACTL6A, NPMI, HDAC2, SOX11, KDM1A, NOC2L, ZBTB9, ZNF697, TRIM32, PPRC1, POU3F1, BNC1, ATF6, OTX1, SSRP1, ETV4, EZH2, DNMT1, PLK4, E2F8, E2F1, DBF4, RAD9A, ZNF280C, DEK, PCGF6, and TDG.

37. The method of any one of claims 31-33, wherein the master regulator of poor prognosis is selected from the group consisting of: SNAI2, E2F7, ARNTL2, LOXL2, HMGA4, MMP14, FOSL1, LIF, FOXD1, LMX1B, TSHZ2, ZNF512B, SNAI1, MEOX2, C2A4RG, MAFK, NCOR2, ZNF532, HADC7, VDR, HTATIP2, NFE2L3, SP100, REST, PLSCR1, FOSL2, TRIM29, and GRHL2.

38. The method of any one of claims 31-33, wherein the master regulator of poor prognosis is selected from the group consisting of: EHF, RBMS1, FLI1, MAFB, SPI1, BASP1, SMARCD3, HAND2, TCFL1, TSHZ3, ZNF521, HEYL, PKNOX2, HIC1, SALL2, KLF17, MEIS3, TGFB1I1, LZTS1, and AEBP1.

39. The method of any one of claims 31-33, wherein the master regulator of poor prognosis is selected from the group consisting of: MYBL2, PTTG1, FOXM1, E2F7, CDK1, UHRF1, TRIP13, TRIM29, HDAC7, ARNTL2, AEBP1, or ACTL6A.

40. The method of any one of claims 31-33, wherein cancer is a cancer type selected from Tables 2 and 3 and the master regulator of poor prognosis is selected from a gene in Tables 2 and 3 and identified as being one of the top 20 master regulators of the cancer type.

41. A method for predicting patient survival in multiple cancer types comprising measuring in a sample from a cancer patient the expression of at least one gene of a master regulator of poor prognosis in the hallmark of epithelial mesenchymal transition pathway selected from the group consisting of: ZNF469, PRRX1, AEBP1, MEIS3, SNAIL MMP14, ADAMTS12, ITGA5, TGFB1I1, and CREB3L1 and comparing the expression with a healthy reference sample, wherein increased expression in the sample from the patient relative to the healthy reference sample is an indicator of reduced predicted survival time.

42. A method for predicting patient survival in multiple cancer types comprising measuring in a sample from a cancer patient the expression of at least one gene of a master regulator of poor prognosis in the reactome cell cycle pathway selected from the group consisting of: MYBL2, CDK1, TRIP13, EZH2, FOXM1, UHRF1, PTTG1, E2F7, BRCA1, and E2F8 and comparing the expression with a healthy reference sample, wherein increased expression in the sample from the patient relative to the healthy reference sample is an indicator of reduced predicted survival time.

43. A method for predicting patient survival in multiple cancer types comprising measuring in a sample from a cancer patient the expression of at least one gene of a master regulator of poor prognosis in the angiogenesis pathway selected from the group consisting of: HEYL, LZTS1, COL4A1, ERG, SOX18, LDB2, GJC1, HLX, SOX17, and PDE3A and comparing the expression with a healthy reference sample, wherein increased expression in the sample from the patient relative to the healthy reference sample is an indicator of reduced predicted survival time.

44. A method for predicting patient survival in multiple cancer types comprising measuring in a sample from a cancer patient the expression of at least one gene of a master regulator of poor prognosis in the immune response pathway selected from the group consisting of: SPI1, IRF1, GATA3, IL2RB, BCL3, FOXP3, ACAP1, GBP1, CXCL13, and WWTR1 and comparing the expression with a healthy reference sample, wherein increased expression in the sample from the patient relative to the healthy reference sample is an indicator of reduced predicted survival time.

45. A method for predicting patient survival in multiple cancer types comprising measuring in a sample from a cancer patient the expression of at least one gene of a master regulator of poor prognosis in the inflammatory response pathway selected from the group consisting of: SPI1, MS4A4A, CIITA, MAFB, VDR, BCL3, LILRB2, IRF5, WWTR1, and CALU and comparing the expression with a healthy reference sample, wherein increased expression in the sample from the patient relative to the healthy reference sample is an indicator of reduced predicted survival time.

46. The method for predicting patient survival comprising measuring in a sample from a cancer patient the expression of at least one gene of a master regulator of poor prognosis and comparing the expression with a healthy reference sample, wherein the cancer is a cancer type as in Tables 2 and 3 and the at least one gene of a master regulator of poor prognosis is selected from a gene in Tables 2 and 3 and identified as being one of the top 20 master regulators of the cancer type, wherein increased expression of the at least one gene of a master regulator of poor prognosis in the sample from the patient relative to the healthy reference sample is an indicator of reduced predicted survival time.

47. A method for treating cancer in a subject with cancer comprising:
(a) obtaining or having obtained a sample from the subject;
(b) measuring or having measured the expression level in the sample of one or more master regulator genes selected from the groups consisting of VDR, CDK1, HDAC7, YAP1, HDAC2, and SMAD7;
(c) comparing the expression level of the one or more master regulators in the sample with the expression level of the one or more master regulators a healthy reference sample, wherein
(i) if VDR expression level is increased in the sample from the subject cancer relative to the healthy reference sample and the subject has GBM, glioma, or AML, administering vitamin D to the subject,
(ii) if CDK1 expression level is increased in the sample from the subject with cancer relative to the healthy reference sample and the subject has lung adenocarcinoma, administering a CDK1/2 inhibitor to the subject,
(iii) if HDAC7 expression level is increased in the sample from the subject with cancer relative to the healthy reference sample and the subject has lung squamous cell carcinoma, colon and/or rectal adenocarcinoma, GBM, or AML, administering an HDAC inhibitor to the subject,
(iv) if YAP1 expression level is increased in the sample from the subject with cancer relative to the healthy reference sample and the subject has pancreatic adenocarcinoma, administering a Yap1 inhibitor to the subject,
(v) if HDAC2 expression level is increased in the sample from the subject with cancer relative to the healthy reference sample and the subject has hepatocellular carcinoma, administering an HDAC inhibitor to the subject, and/or
(vi) if SMAD7 expression level is increased in the sample from the subject with cancer relative to the healthy reference sample and the subject has lung squamous cell carcinoma, administering mongersen and/or a TGFbeta pathway inhibitor to the subject.

48. The method of claim 47, wherein the CDK1/2 inhibitor is flavopiridol; the HDAC inhibitor is vorinostat, romidepsin, belinostat, panobinostat, entinostat, or valproic acid; the Yap1 inhibitor is vereporfin, CA3, trametinib, dasatinib, or metformin; and the TGFbeta pathway inhibitors is galunisertib or AVID200.
